(19) United States
(12) Patent Application Publication
(10) Pub. No.: US 2025/0041492 A1
(43) Pub. Date: Feb. 6, 2025

(54) CRYOPRECIPITATE SYSTEMS AND METHODS OF USING THE SAME

(71) Applicant: Terumo BCT, Inc., Lakewood, CO (US)

(72) Inventors: Dennis HLAVINKA, Lakewood, CO (US); John PITTINGER, Evergreen, CO (US)

(21) Appl. No.: 18/786,959

(22) Filed: Jul. 29, 2024

Related U.S. Application Data
(60) Provisional application No. 63/529,815, filed on Jul. 31, 2023.

(51) Int. Cl. A61M 1/02

(52) U.S. Cl. CPC A61M 1/0286; A61M 1/0209; A61M 1/0281; A61M 2202/0415

(57) ABSTRACT

A shelfing structure for use during the precipitation of one or more components from a composite includes a support and one or more expression plates movably coupled to the support, where each of the one or more expression plates is movable between a first position and a second position, and each of the one or more expression plates is configured to receive one or more bags of a bag assembly. A method for separating one or more components from a composite may include positioning the shelfing structure holding one or more bag assemblies in an environment having a thawing temperature. Each of the one or more bag assemblies may include a first bag including the composite in a frozen state and a second bag configured to receive the one or more components. The shelfing structure may be configured to hold the second bag above the first bag in each instance.

### CROSS-REFERENCE TO RELATED APPLICATIONS

This application claims the benefit of U.S. Provisional Application No. 63/529,815 filed on Jul. 31, 2023. The entire disclosure of the above application is incorporated herein by reference.

### FIELD

The present disclosure relates to systems and methods for the separation of one or more components from composites.

### BACKGROUND

This section provides background information related to the present disclosure which is not necessarily prior art.

Cryoprecipitate antihemophilic factor, which is commonly referred to simply as cryoprecipitate, is a portion of blood plasma (i.e., the liquid part of whole blood) that is rich in clotting factors. Clotting factors are proteins that can reduce blood loss by helping to slow or stop bleeding. The clotting factors of cryoprecipitate plays an important role in various therapies and treatments for subjects or patients whose own blood does not properly clot, whether because of genetic differences or illness or injury.

Cryoprecipitate is often prepared (i.e., separated from blood plasma) by thawing frozen plasma to generate a cryoprecipitate and centrifuging the thawed plasma to sediment the cryoprecipitate away from cryoprecipitate-poor plasma (CPP). The cryoprecipitate may then be collected. Collecting the cryoprecipitate may include one or more draining processes and one or more measuring processes, for example, to redistribute some of the cryoprecipitate-poor plasma back to the sedimented cryoprecipitate. After the cryoprecipitate is isolated, the methods often include pooling several cryoprecipitate units together and re-freezing the pooled cryoprecipitate for storage for future use for various therapies and treatments.

It would be desirable to improve systems for and methods of preparing, or manufacturing, cryoprecipitate that reduce the amount of time and labor often required for separating and pooling cryoprecipitate, while also improving the consistency of the collected cryoprecipitate.

### SUMMARY

This section provides a general summary of the disclosure and is not a comprehensive disclosure of its full scope or all of its features.

At least one example embodiment relates to a shelfing structure for use during the precipitation of one or more components from a composite.

In at least one example embodiment, the shelfing structure may include a support and one or more expression plates movably coupled to the support, where each of the one or more expression plates is movable between a first position and a second position, and each of the one or more expression plates is configured to receive one or more bags of a bag assembly.

In at least one example embodiment, the shelfing structure may further include one or more spring loaded hinges movably coupling each of the one or more expression plates and the support.

In at least one example embodiment, each of the one or more expression plates may include a first portion and a second portion, where the second portion is movably coupled to the first portion to define the first position and the second position.

In at least one example embodiment, the shelfing structure may further include one or more locking mechanisms, where the one or more locking mechanism are configured to maintain the one or more expression plates in the first position.

In at least one example embodiment, the support may further include a plurality of castors.

In at least one example embodiment, each of the one or more expression plates may have a first surface opposing a second surface. A first distance between the first surface and the support may define the first position. A second distance between the first surface and the support that is less than the first distance may define the second position.

In at least one example embodiment, the bag assembly may include a first bag and a second bag. The first surface may be configured in each instance to support the first bag.

In at least one example embodiment, the first surface may include one or more guiders that are configured to help position the first bag relative to the first surface.

In at least one example embodiment, the second surface may be configured in each instances to support the second bag.

In at least one example embodiment, the second surface may include one or more guiders that are configured to help position the second bag relative to the second surface.

In at least one example embodiment, the second surface may be configured to support the second bag above the first bag.

In at least one example embodiment, the bag assembly may further include a tube length connecting the first bag and the second bag.

In at least one example embodiment, the tube length may include a line clamping member.

In at least one example embodiment, the shelfing structure may further include a clamping structure, where the clamping structure is configured to clamp the tube length.

In at least one example embodiment, the first surface may include one or more stoppers that are configured to engage with the support to define the second position.

At least one example embodiment relates a bag assembly for pooling one or more components as separated from two or more composites.

In at least one example embodiment, the bag assembly may include a first bag subassembly including a first bag, a second bag, and a first tube length connecting the first bag and the second bag; a second bag subassembly including a third bag, a fourth bag, and a second tube length connecting the third bag and the fourth bag; and a single bag in fluid communication with the second bag of the first bag subassembly and the fourth bag of the second bag subassembly.

In at least one example embodiment, the first tube length may include a first line clamp configured to move between a first position and a second position. In the first position, the first line clamp may occlude the first tube length and prevent material movement between the first bag and the second bag. In the second position, the first clamp may allow material movement between the first bag and the second bag.

In at least one example embodiment, the first bag, the first tube length, and the second bag may be integrally formed.

In at least one example embodiment, the second tube length may include a second line clamp configured to move between a first position and a second position. In the first position, the second line clamp may occlude the second tube length and prevent fluidic movement between the third bag and the fourth bag. In the second position, the second line clamp may allow fluidic movement between the third bag and the fourth bag.

In at least one example embodiment, the third bag, the second tube length, and the fourth bag may be integrally formed.

In at least one example embodiment, the bag assembly may further include a third tube length connecting the single bag and the second bag of the first subassembly and also the single bag and the fourth bag of the second bag subassembly.

In at least one example embodiment, the second bag of the first subassembly, the fourth bag of the second bag subassembly, the third tube length, and the single bag may be integrally formed.

In at least one example embodiment, the third tube length may include a third clamp configured to move between a first position and a second position. In the first position, the third clamp may occlude the third tube length and prevent material movement between the single bag and the second bag of the first subassembly and the fourth bag of the second bag subassembly. In the second position, the third clamp may allow material movement between the single bag and the second bag of the first subassembly and the fourth bag of the second bag subassembly.

In at least one example embodiment, the bag assembly may further include a fourth tube length fluidically connecting the second bag of the first subassembly and the third tube length.

In at least one example embodiment, the fourth tube length may include a third clamp configured to move between a first position and a second position. In the first position, the third clamp may occlude the fourth tube length and prevent material movement between the second bag of the first subassembly and the third tube length. In the second position, the third clamp may allow material movement between the second bag of the first subassembly and the third tube length.

In at least one example embodiment, the bag assembly may further include a fifth tube length fluidically connecting the fourth bag of the second subassembly and the third tube length.

In at least one example embodiment, the fifth tube length may include a fourth clamp configured to move between a first position and a second position. In the first position, the fourth clamp may occlude the fifth tube length and prevent material movement between the fourth bag of the second subassembly and the third tube length. In the second position, the fourth clamp may allow material movement between the fourth bag of the second subassembly and the third tube length.

In at least one example embodiment, at least one of the second bag of the first subassembly and the fourth bag of the second subassembly may have as funnel shaped portion disposed towards the single bag.

In at least one example embodiment, the first tube length may include a sedimentation filter.

At least one example embodiment relates to a method for separating one or more components from a composite.

In at least one example embodiment, the method includes positioning a shelfing structure holding one or more bag assemblies in an environment having a predetermined or selected thawing temperature. Each of the one or more bag assemblies may include a first bag including the composite in a frozen state and a second bag configured to receive the one or more components. The shelfing structure may be configured to hold the second bag above the first bag in each instance.

In at least one example embodiment, the shelfing structure may include a support and one or more expression plates movably coupled to the support. Each of the one of the one or more expression plates may have a first surface opposing a second surface. The first surface may be configured to support the first bag of a first bag assembly of the one or more bag assemblies. The second surface may be configured to support the second bag of a second bag assembly of the one or more bag assemblies.

In at least one example embodiment, the shelfing structure may further include one or more spring loaded hinges that movably couple each of the one or more expression plates and the support.

In at least one example embodiment, each of the one or more expression plates may be movable between a first position and a second position in response to the thawing of the frozen state of the composite.

In at least one example embodiment, the shelfing structure may further include one or more locking mechanisms, where the one or more locking mechanisms are configured to maintain the one or more expression plates in the first position, and the method may further include adjusting the one or more locking mechanism prior to, concurrently with, or subsequent to the positioning of the shelfing structure holding the one or more bag assemblies into the environment having the predetermined or selected thawing temperature.

In at least one example embodiment, each of the one or more bag assemblies may include a first tube length connecting the first bag and the second bag. The first tube length may include a line clamp configured to move between a first position and a second position. In the first position, the line clamp may occlude the first tube length and prevent fluidic movement between the first bag and the second bag. The method may further include adjusting the line clamp prior to, concurrently with, or subsequent to the positioning of the structure holding the one or more bag assemblies into the environment having the predetermined or selected thawing temperature.

In at least one example embodiment, the method may further include joining together the first bag and the second bag via the first tube length.

In at least one example embodiment, each of the one or more bag assemblies may be connected to a third bag.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1A is a side view of an example shelfing structure for use during the freezing and thawing of a composite including one or more components to prepare a precipitate, where the shelfing structure includes a plurality of shelves that are configured to occupy several positions and to receive one or more bags that include the composite, and where the plurality of shelves occupy a first position, according to at least one example embodiment;

FIG. 1B is a side view of the example shelfing structure as illustrated in FIG. 1A where the plurality of shelves occupy a second position to exert a first force or pressure onto the one or more bags according to at least one example embodiment;

FIG. 1C is a side view of the example shelfing structure as illustrated in FIG. 1A where the plurality of shelves occupy a third position to exert a second force or pressure onto the one or more bags according to at least one example embodiment;

FIG. 1D is a front view of the example shelfing structure as illustrated in FIG. 1A where the plurality of shelves occupy the first position according to at least one example embodiment;

FIG. 1E is a front view of the example shelfing structure as illustrated in FIG. 1A where the plurality of shelves occupy the second position according to at least one example embodiment;

FIGS. 2A-2C are illustrations of an individual shelf of the plurality of shelves of the example shelfing structure illustrated in FIGS. 1A-IE, where FIG. 2A shows the individual shelf in the first position according to at least one example embodiment, FIG. 2B shows the individual shelf in the third position according to at least one example embodiment, and FIG. 2C shows the individual shelf in the second position according to at least one example embodiment;

FIG. 3A is a side view of another example shelfing structure for use during the freezing and thawing of a composite including one or more components to prepare a precipitate, where the shelfing structure includes a plurality of shelves that are configured to occupy several positions and to receive one or more bags that include the composite, and where the plurality of shelves occupy a first position, according to at least one example embodiment;

FIG. 3B is a side view of the example shelfing structure as illustrated in FIG. 3A where the plurality of shelves occupy a second position to exert a first force or pressure onto the one or more bags according to at least one example embodiment;

FIG. 3C is a side view of the example shelfing structure as illustrated in FIG. 3A where the plurality of shelves occupy a third position to exert a second force or pressure onto the one or more bags according to at least one example embodiment;

FIG. 3D is a front view of the example shelfing structure as illustrated in FIG. 3A where the plurality of shelves occupy the first position according to at least one example embodiment;

FIG. 3E is a front view of the example shelfing structure as illustrated in FIG. 3A where the plurality of shelves occupy the second position according to at least one example embodiment;

FIGS. 4A-4C are illustrations of an individual shelf of the plurality of shelves of the example shelfing structure illustrated in FIGS. 3A-3E, where FIG. 4A shows the individual shelf in the first position according to at least one example embodiment, FIG. 4B shows the individual shelf in the third position according to at least one example embodiment, and FIG. 4C shows the individual shelf in the second position according to at least one example embodiment;

FIG. 5A is an illustration of an example bag assembly for use during the freezing and thawing of a composite including one or more components, for example, for use with the example shelfing structure illustrated in FIGS. 1A-1E and/or the example shelfing structure illustrated in FIGS. 3A-3E, according to at least one example embodiment;

FIG. 5B is an illustration of another example bag assembly for use during the freezing and thawing of a composite including one or more components, for example, for use with the example shelfing structure illustrated in FIGS. 1A-1E and/or the example shelfing structure illustrated in FIGS. 3A-3E, according to at least one example embodiment;

FIG. 5C is an illustration of another example bag assembly for use during the freezing and thawing of a composite including one or more components, for example, for use with the example shelfing structure illustrated in FIGS. 1A-1E and/or the example shelfing structure illustrated in FIGS. 3A-3E, according to at least one example embodiment;

FIG. 6 is a flowchart illustrating an example method for the separation of one or more components from a composite using, for example, the example shelfing structure illustrated in FIGS. 1A-1E and/or the example shelfing structure illustrated in FIGS. 3A-3E and/or one or more of the example bag assemblies illustrated in FIGS. 5A-5C, according to at least one example embodiment;

FIG. 7 is a flowchart illustrating another example method for the separation of one or more components from a composite using, for example, the example shelfing structure illustrated in FIGS. 1A-1E and/or the example shelfing structure illustrated in FIGS. 3A-3E and/or one or more of the example bag assemblies illustrated in FIGS. 3A-3C, according to at least one example embodiment; and

FIGS. 8A-8H are pictorials of an example method for pooling a separated precipitate using, for example, the example structure illustrated in FIGS. 1A-IE and/or the example shelfing structure illustrated in FIGS. 3A-3E and/or one or more of the example bag assemblies illustrated in FIGS. 5A-5C and/or the example methods illustrated in FIGS. 6 and 7, according to at least one example embodiment.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

Example embodiments will now be described more fully with reference to the accompanying drawings.

In various aspects, shelfing structures or racks for use during the freezing and thawing of a composite including one or more components to prepare a precipitate, where the shelfing structure includes a plurality of shelves (or expression plates) that are configured to occupy several positions and to receive one or more bags that include the composite, are provided. The plurality of shelves may be configured to receive the one or more bags that include the composite and to move from an extended (or first) position (or state) to a retracted (or second) position (or state), where in the extended position the plurality of shelves hold the one or more bags but do not apply a force or pressure thereon and in the retracted position the plurality of shelves hold the one or more bag and apply a force or pressure thereon. In the extended position, the plurality of shelves may be locked to hold the one or more bags during freezing of the composite. In the retracted position, the plurality of shelves may be unlocked to hold the one or more bags in a manner to express thawed material from the one or more bags. The shelfing structures may be used for the freezing and thawing of blood plasma to prepare a cryoprecipitate.

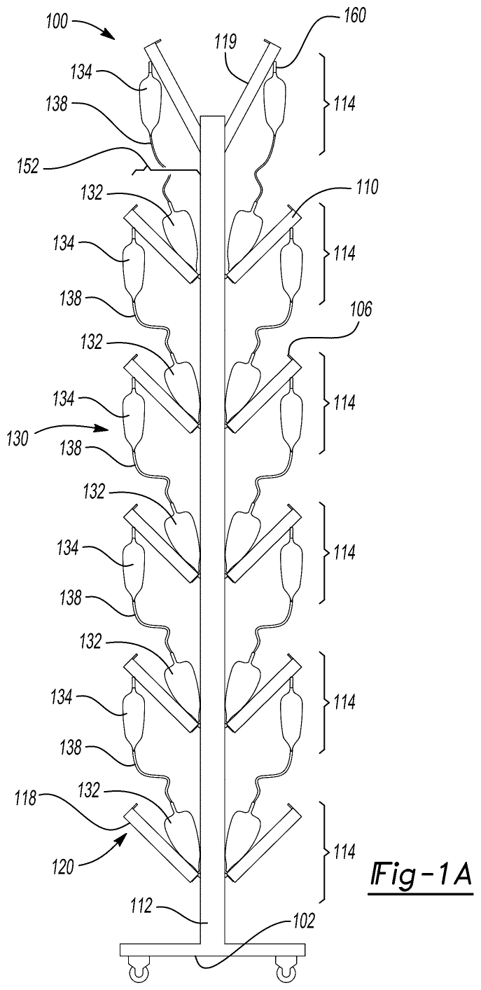

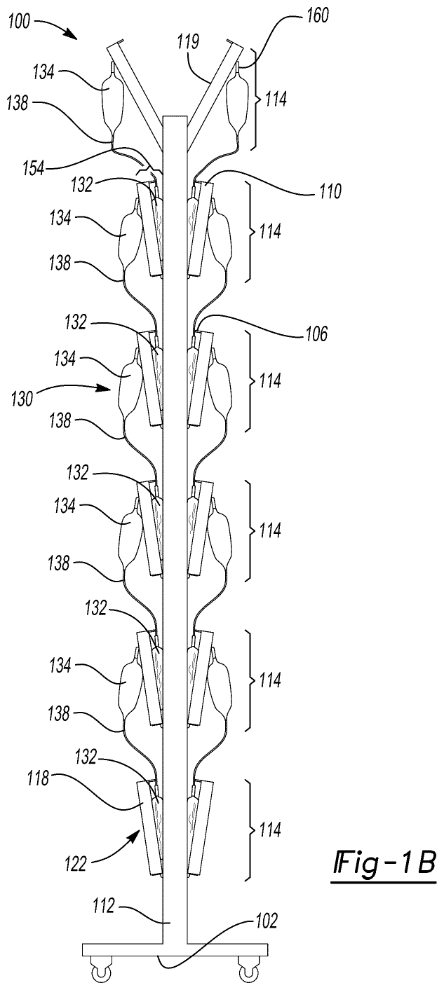

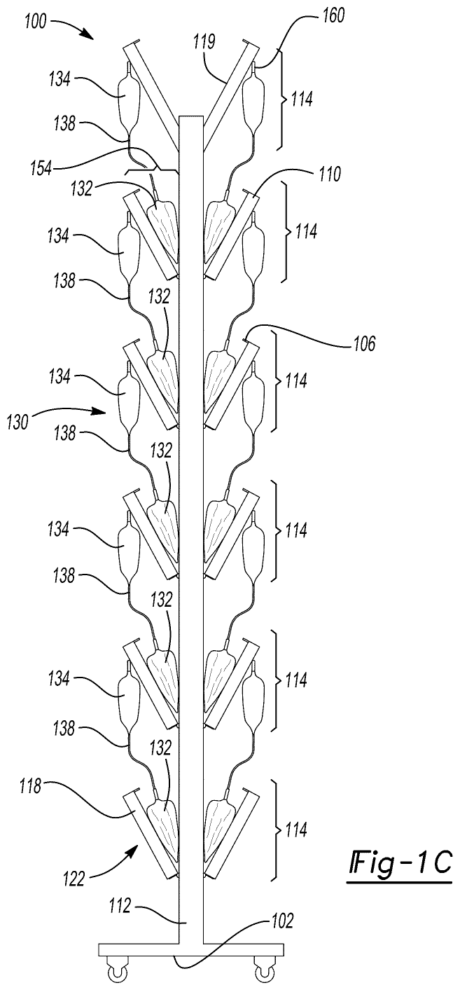

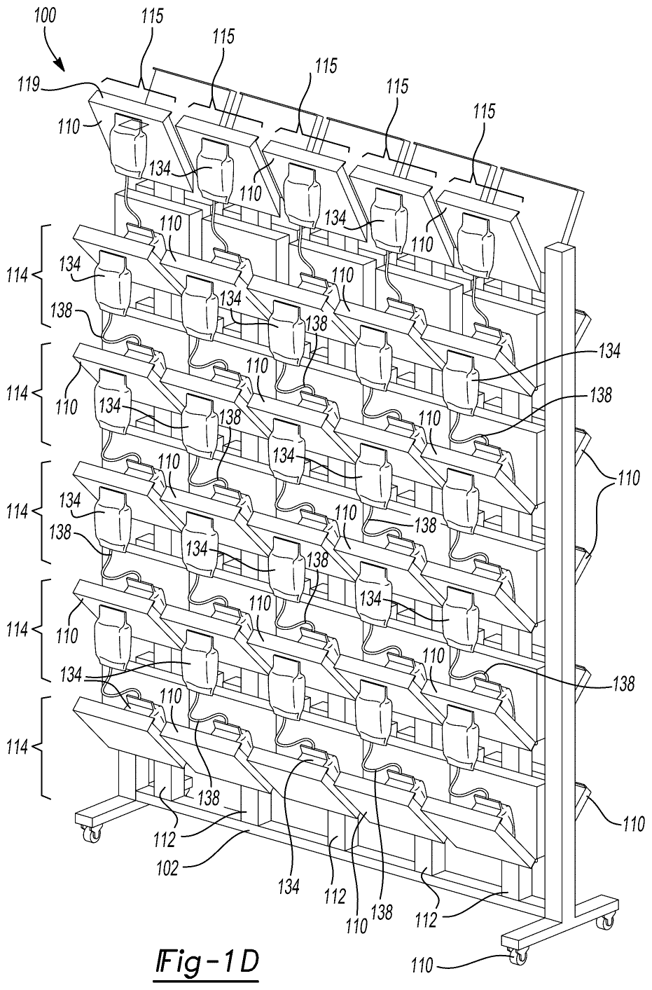

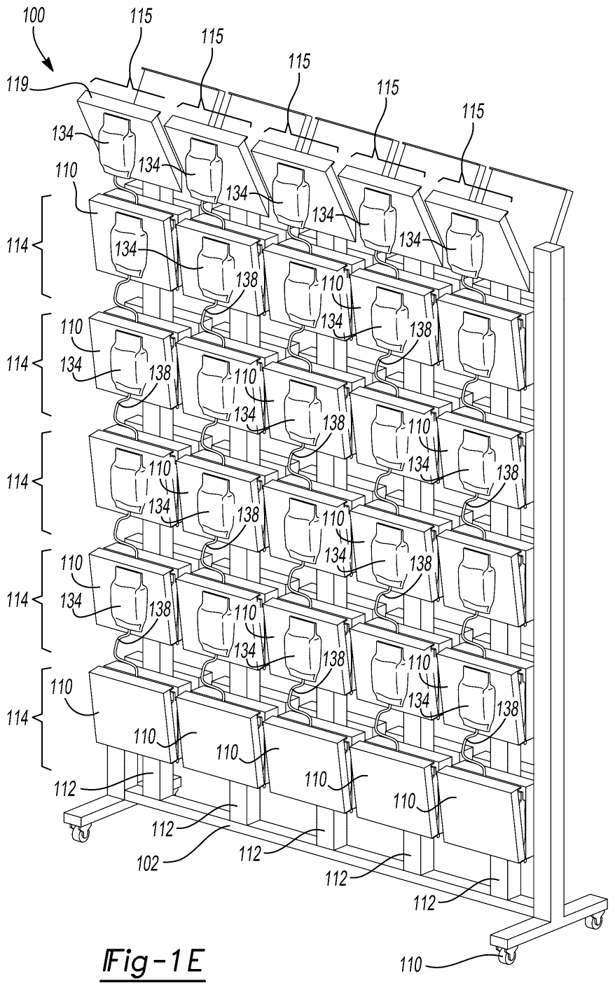

FIG. 1A is a side view of an example shelfing structure 100 for use during the freezing and thawing of a composite including one or more components to prepare a precipitate, where the shelfing structure 100 includes a plurality of shelves (or expression plates) 110 and the plurality of shelves 110 are in an extended (or first or open) position (or state) 120. FIG. 1B is another side view of the shelfing structure 100, where the plurality of shelves 110 are in a retracted (or second or closed) position (or state) 122. FIG. 1C is another side view of the shelfing structure 100, where the plurality of shelves 110 are in an intermediate (or third) position (or state) 124 between the extended position and the retracted position. FIG. 1D is a front view of the shelfing structure 100, where the plurality of shelves 110 are in the extended position. FIG. 1E is another front view of the shelfing structure 100, where the plurality of shelves are in the retracted position. In at least one example embodiment, the composite may include plasma, and the structure 100 may be used during the freezing and thawing of blood plasma to isolate cryoprecipitate therefrom.

The shelfing structure 100 may include one or more rows 114, where each row 114 includes one or more of the plurality of shelves 110 and each of the one or more shelves 110 may be configured to receive or support a composite-containing bag (or container) 132. For example, in at least one example embodiment, as illustrated, the shelfing structure 100 may include five rows 114 and five columns 115. Although individual shelves 110 are illustrated as receiving individual bags (or containers) 132, it should be appreciated that, in various other example embodiments, at least one of the one or more rows 114 may be defined by a single shelf 110 that receives two or more composite-containing bags 132. Although not specifically illustrated, it should be appreciated that, in various other example embodiments, one or more of the shelves 110 of the shelfing structure 100 and/or the support 112 of the shelfing structure 100 may include one or more layers or coatings of insulation on one or more surfaces.

The shelfing structure 100 may include a plurality of supports 112. Each of the supports 112 may have a first end and an opposing second end, where the first end of the supports 112 are coupled to a base 102. The supports 112 may be substantially perpendicular to the base 102. The base 102 may include one or more castors 111, or like structures, to allow the shelfing structure 100 to be easily positioned, including, for example, moved between different environments. In at least one example embodiment, the castors 111 may include lockable castors.

Each of the plurality of shelves 110 may include a first end and an opposing second end, where the first end of the respective shelf 110 may be individually and movably coupled to a respective support 112. Each of the plurality of shelves 110 may be coupled to the respective support 112 in a manner that allows the respective shelves 110 to be individually displaced relative to the support 112. For example, in at least one example embodiment, the shelves 110 may be movably coupled to the respective support 112 using, for example, spring loaded hinges 126.

Each of the plurality of shelves 110 may be individually movable relative to the respective support 112. For example, the shelves 110 may be individually and independently movable between the extended position 120 (which is illustrated in FIGS. 1A and 1D) and the intermediate position 124 (which is illustrated in FIG. 1C) and between the intermediate position 124 and the retracted position 122 (which is illustrated in FIGS. 1B and 1E) and between the extended position 120 and the retracted position 122. In the extended position 120, a first space 152 may be defined between the shelf 110 and the support 112. In the retracted position 122, a second space 154 that may be smaller than the first space 152 may be defined between the shelf 110 and the support 112. In the intermediate position 124, a third space 156 that may be smaller than the first space 152 and larger than the second space 154 may be defined between the shelf 110 and the support 112.

In at least one example embodiment, one or more of the plurality of shelves 110 may include one or more stoppers (or spacers) 106. The one or more stoppers 106 may extend from a first surface (or first side or top side or top surface) 116 of the shelf 110 and may engage (or interact) with the support 112 to define the retracted position 122 and the second space 154. In at least one example embodiment, the one or more stoppers 106 may be configured (for example, a length determined) in response to a determined or desired fluid to remain in the composite-containing bag (or container) 132 after a thaw cycle.

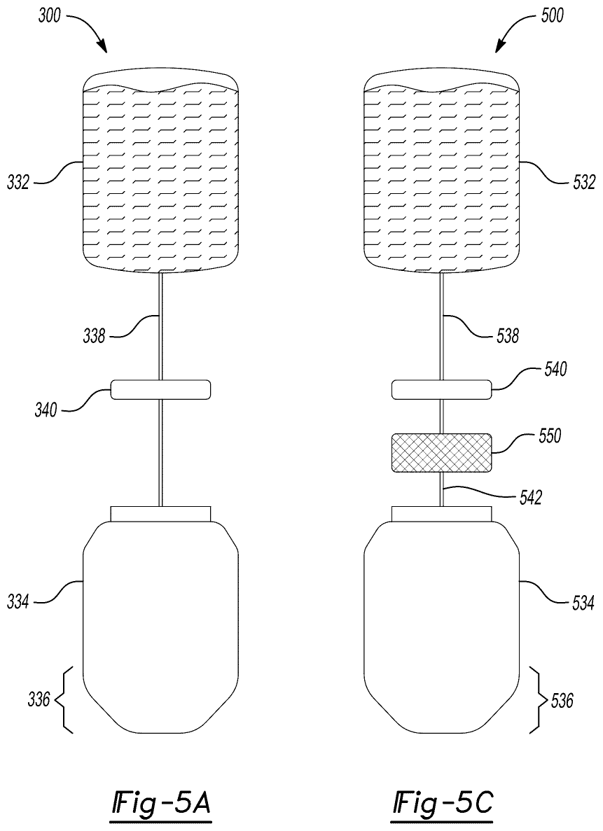

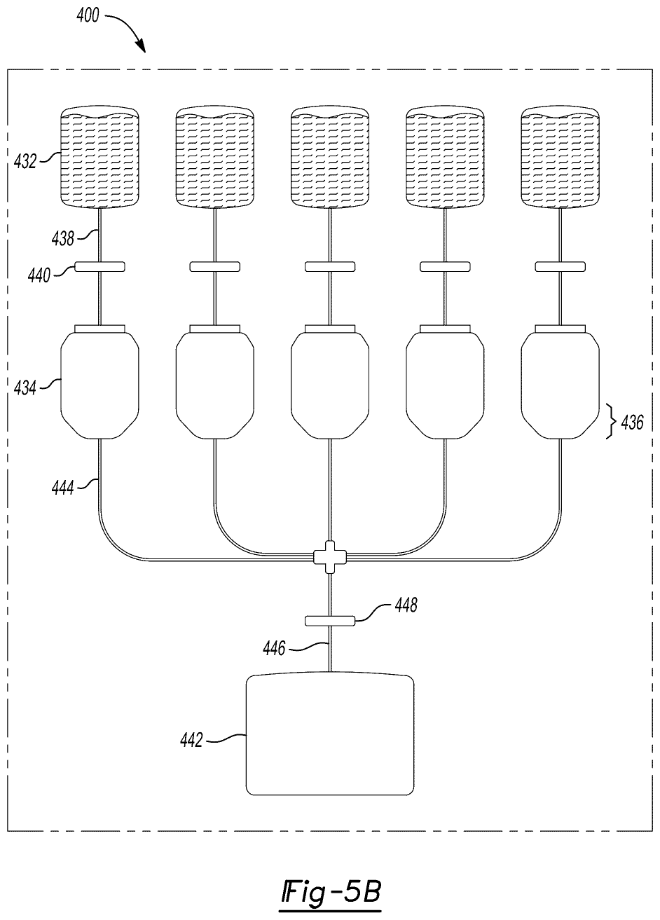

The shelfing structure 100 receives or supports one or more bag or container assemblies 130. In at least one example embodiment, each shelf of the plurality of shelves 110 may be associated with at least one bag assembly 130. Each of the bag assemblies 130 may include a first bag or container 132 and a second bag or container 134 in communication therewith. For example, in at least one example embodiment, a tube length 138 may fluidically connect the first bag 132 and the second bag 134. Although not illustrated, it should be appreciated that, in various example embodiments, the bag assembly 130 may include one or more ports (for example, for sterile docking) for previous or subsequent connections with other sources and/or one or more line clamps for controlling flow between the first and second bags 132, 134 and/or one or more filters disposed along the tube length 128. Further, although not illustrated in this instance, it should be appreciated that, in various example embodiments, the bag assembly 130 may include one or more other bags (for example, a third bag 442 as suggested in FIG. 5B).

The first bag 132 may include a composite-containing bag or container (e.g., a plasma source bag or container) that is supported by the respective shelf 110, while the second bag 134 may include a thaw bag or container configured to receive unselected components of the composite, such as cryoprecipitate-poor plasma. The first bag 132 may be associated with and supported by the first surface 116 of a first shelf of the plurality of shelves 110, while the second bag 134 may be associated with a second surface 117 of a second shelf of the plurality of shelves 110 or another structure 119 of the shelfing structure 110, where the second shelf or the another structure 119 is disposed above the first shelf. In at least one example embodiment, the second bag 134 may be hung from the second shelf or the another structure 119. The another structure 119 may be a top row or shelf of the shelfing structure 100 that is configured to receive or support the second bag 134 but that does not receive or support the first bag 132.

Although not illustrated, it should be appreciated that, in various example embodiments, the shelfing structure 100, and more specifically at least one of the first shelf of the plurality of shelves 100, the second shelf of the plurality of shelves 100, and the another structure 119, may be configured to support one or more other bags (for example, a third bag 442 as suggested in FIG. 5B). In at least one example embodiment, the third bag may be clamped to one of the first shelf of the plurality of shelves 100, the second shelf of the plurality of shelves 100, and the another structure 119. In other example embodiments, the third bag 442 may be hung from one of the first shelf of the plurality of shelves 100, the second shelf of the plurality of shelves 100, and the another structure 119.

Although not illustrated, it should be appreciated that, in various example embodiments, the shelfing structure 100 may include one or more holding or locator components that are configured to help guide positioning of the first and second bags 132, 134. For example, in at least one example embodiment, the first surface 116 of the respective shelves 110 may include one or more locator pins for guiding the placement of the first bag 132. In at least one example embodiments, the second surface 117 of the respective shelves 110 and also the another structure 119 may include one or more hooks for receiving and supporting the second bags 134. In at least one example embodiment, the one or more hooks may be configured to receive an angled portion of the second bag 134.

In at least one example embodiment, the shelves 110 may be locked in the extended position 120 during freezing of the composite (or during receipt of a frozen composite), and while in the unlocked position, the shelves 110 may be permitted to move in response to the thawing state of the composite. For example, although not illustrated, it should be appreciated that, in various example embodiments, the shelfing structure 100 may include one or more locking mechanisms as selected by the skilled artisan to hold the shelves 110 in the extended position. In at least one example embodiment, the one or more locking mechanisms may include a latching mechanism.

The shelves 110 of the shelfing structure 100 may be locked in the extended position 120 to receive the first bag 132 in a frozen state or to receive the first bag 132 holding a composite to be frozen. At the beginning, or during, thawing of the frozen state, the shelves 110 may be unlocked, manually or using automatic assembly, and allowed to move from the extended position 120 to the retreated position 122 and/or form the extended position 120 to the intermediate position 124 and/or from the intermediate position 124 to the retracted position 122. As the frozen state thaws and the shelves 110 may move from the extended position 120 to the retracted position 122 and/or from the extended position 120 to the intermediate position 124 and/or form the intermediate position 124 to the retracted position 122, the shelves 110 (for example, via the spring loaded hinges 126) may apply a force or pressure to the first bag 132 that is sufficient to cause the movement of thawed liquid from the first bag 132 to the second bag 134, for example, via the tube length 138. In this manner, the relative state of the first bag 132 may control the relative position (or state) of the respective shelf 110.

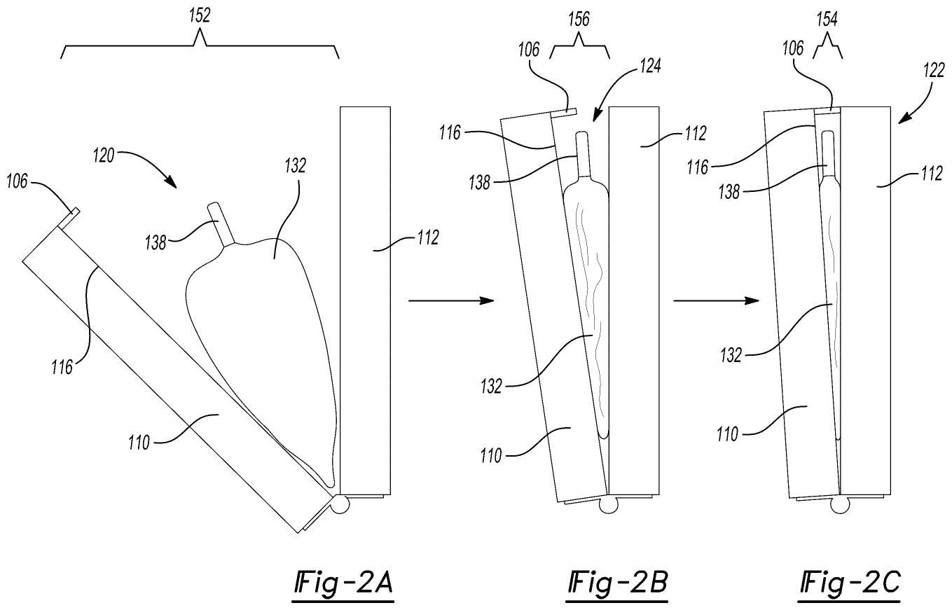

FIGS. 2A-2C are illustrations of an individual shelf 110 of the shelving structure 100, where in FIG. 2A the shelf 110 is in the extended position 120 where the shelf 110 is associated with (e.g., supports) the first bag 132 and the first bag 132 includes a frozen composite, in FIG. 2B the shelf 110 is in the intermediate position 124 where the shelf 110 is associated with (e.g., supports) the first bag 132 and the first bag 132 includes a frozen composite during a thawing process, and in FIG. 2C the shelf 110 is in the retracted position 122 where the shelf 110 is associated with (e.g., supports) the first bag 132 and the first bag 132.

In at least one example embodiment, as illustrated in FIG. 2A, the shelves 110 of the shelfing structure 100 may be held in the first position 120 during freezing (or during receipt of a composite to be frozen). In the first position 120, at least one of the shelves 110 may receive the first bag 132, which includes liquid plasma. The liquid plasma may be frozen while held or supported by the respective shelf 110. That is, the frozen plasma may be shaped by the position of the respective shelf 110. Before, or during, subsequent thawing, as illustrated in FIG. 2B, the shelves 110 may be unlocked and the respective shelf 110 may apply a force or pressure on the first bag 132. The force or pressure of the shelf 110 on the respective bag 132 may cause non-precipitate to move to the second bag 134 at a rate that is equivalent to the rate of the melting of the frozen plasma, where cryoprecipitate remains in the first bag 132 after thawing, as illustrated in FIG. 2C.

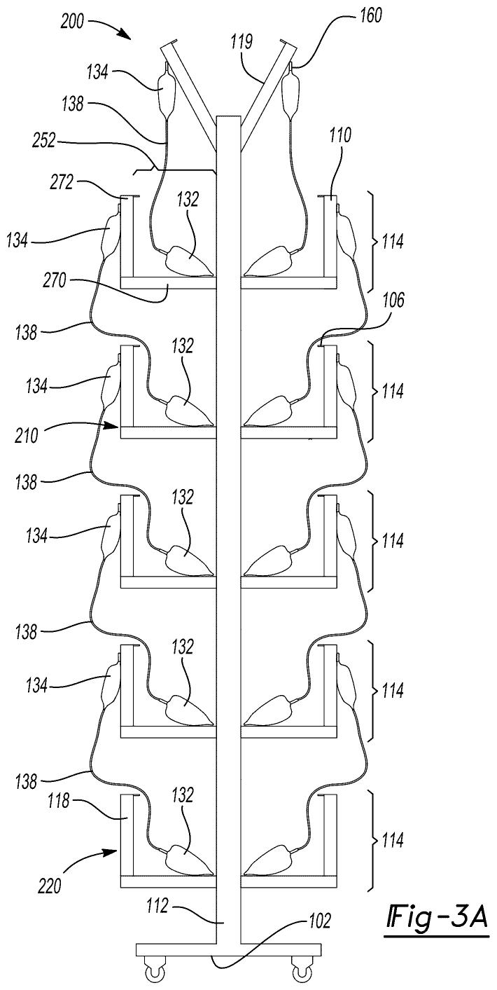

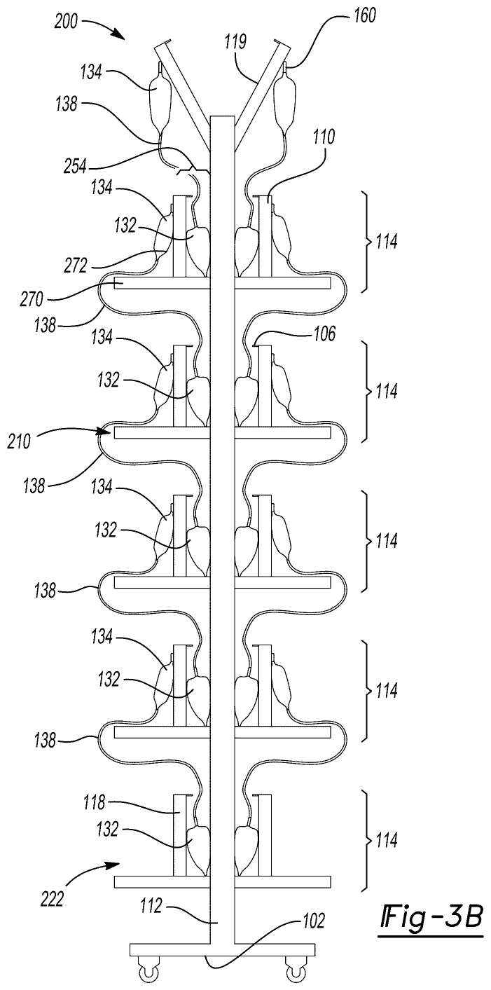

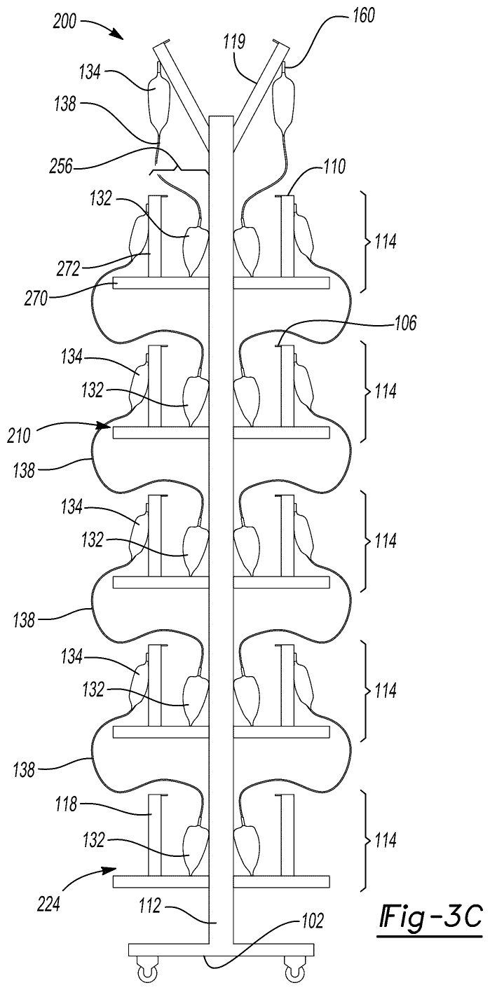

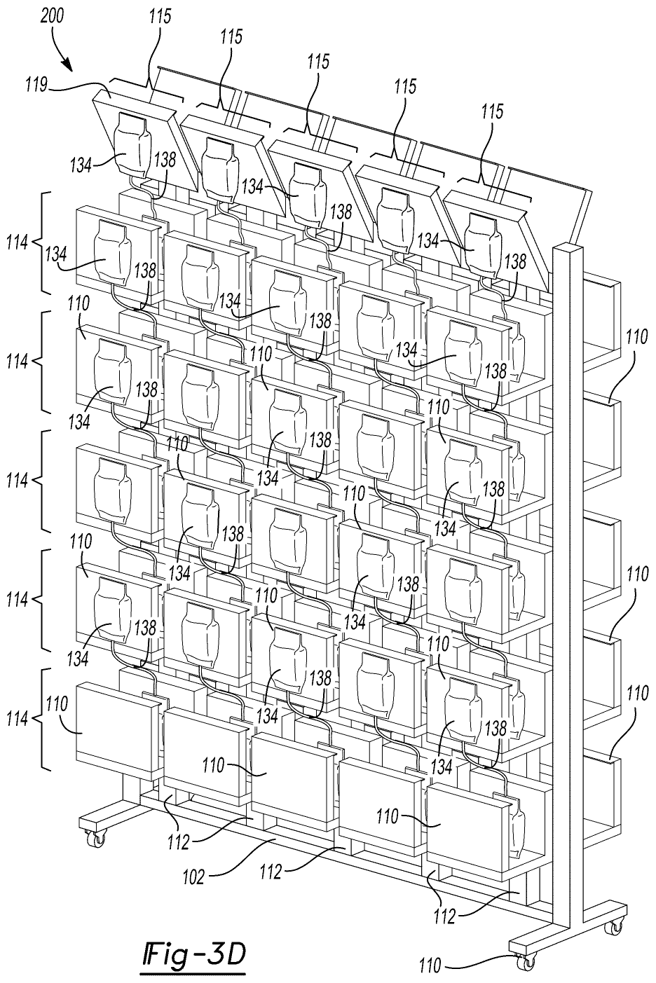

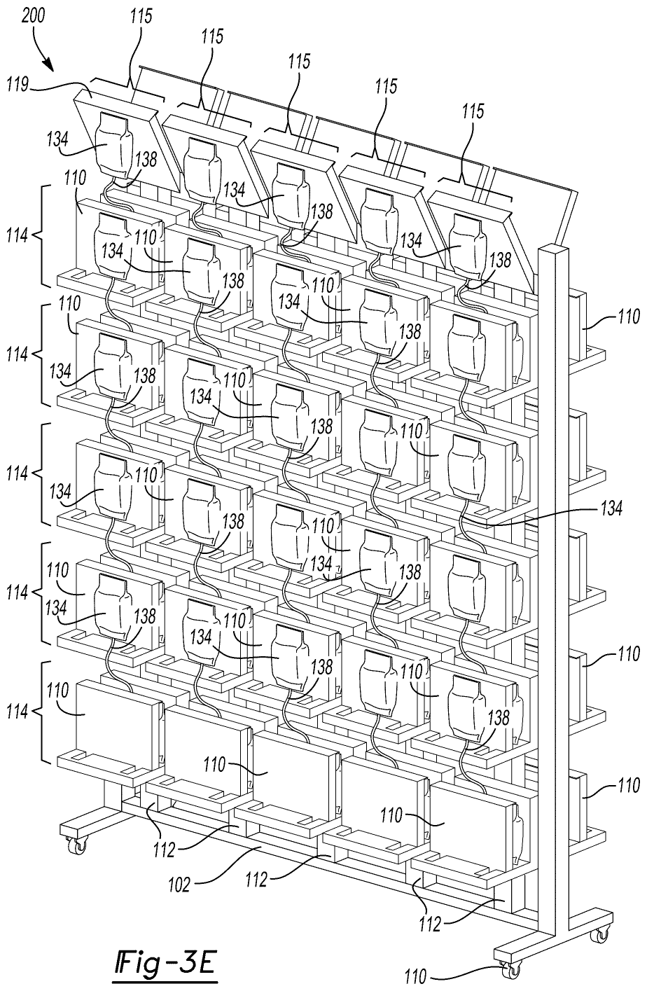

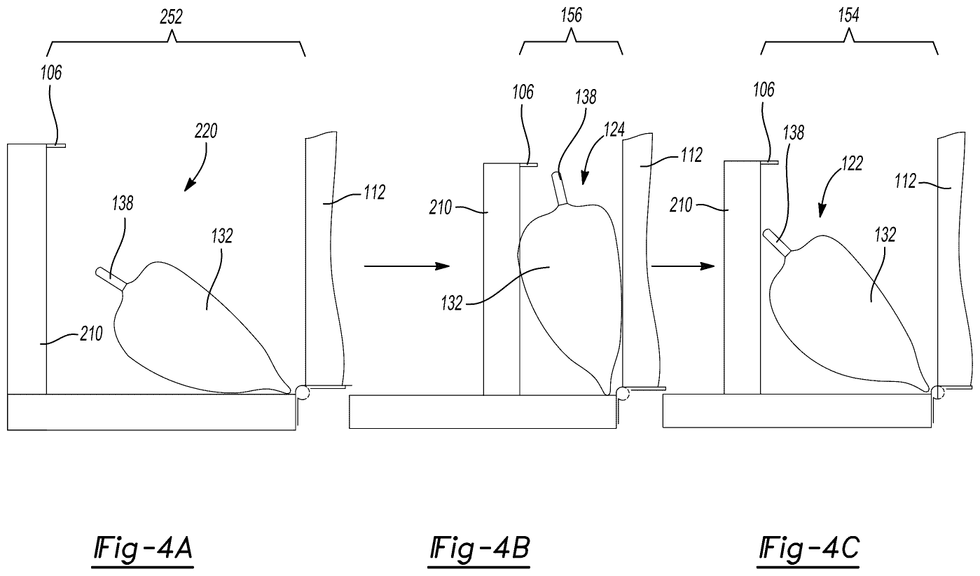

Transitioning from the solid or frozen state to the liquid state may occur at a constant temperature, for example, about 4° C. In at least one example embodiment, a constant temperature may be assured by moving the shelving structure 100 into a refrigerator. The thawing process may also be controlled as needed, for example, by varying the compositional structure of one or more shelves 110 of the shelfing structure 100 and/or the support 112 of the shelfing structure. For example, in at least one example embodiment, one or more of the plurality of shelves 110 of the shelfing structure 100 and/or the support 112 of the shelfing structure 100 may include one or more insulating layers or coatings. The thawing process may also be controlled as needed, for example, by using one or more fans. For example, although not illustrated, it should be appreciated that, in various example embodiments, one or more of the plurality of shelves 110 and/or the another structure 119 may include one or more fans or fan components. For example, the one or more fans or fan components may be embedded within the structure of the one or more of the plurality of shelves 110 and/or the another structure 119 (or in the instance of FIGS. 3A-4C, in the first (or stationary or horizontal) portion 270 and/or the second (or movable or vertical) portion 272). In at least one example embodiment, the structure 100, and more particularly, the individual fans or fan components, may be in communication with a control system that may help to ensure a desired temperature of the plates (for example, that freezing and melting occurs at a selected or predetermined rate). Further, although not illustrated, it should be appreciated that, in various example embodiments, one or more of the plurality of shelves 110 and/or the another structure 119 may include one or more finned heat exchangers or components of one or more finned heat exchangers. For example, the one or more finned heat exchangers or components of one or more finned heat exchangers may be embedded within the structure of the one or more of the plurality of shelves 110 and/or the another structure 119 (or in the instance of FIGS. 3A-4C, in the first (or stationary or horizontal) portion 270 and/or the second (or movable or vertical) portion 272). In at least one example embodiment, the structure 100, and more particularly, the individual finned heat exchangers or components of one or more finned heat exchangers, may be in communication with a control system that may help to ensure a desired temperature of the plates (for example, that freezing and melting occurs at a selected or predetermined rate). In each instance, assuring that thawing occurs at the constant temperature may help to keep precipitates from going back into solution, which can help to improve overall yields of the one or more components (e.g., cryoprecipitate).

Additionally, or alternatively, removing the liquid from the first bag 132 during the thawing process may reduce the time necessary for the thawing process. For example, removing the liquid from the first bag 132 during the thawing process may help to assure that all heat, from the relatively warmer refrigerator, entering the first bag 132 is directed to the frozen state and not warming the liquid or melted state. That is, received heat does not need to pass through the liquid to reach the remaining ice. Further, removing the liquid from the first bag 132 during the thawing process may help to increase and/or maintain the surface area of the first bag 132 interfacing with the expression plate 110 and/or support 112, improving heat transfer.

FIGS. 3A-3E illustrate another example shelfing structure 200 for use during the freezing and thawing of a composite including one or more components to prepare a precipitate. The shelfing structure 200 as illustrated in FIGS. 3A-3E is the same as the shelfing structure 100 as illustrated in FIGS. 1A-IE expect that the plurality of shelves 210 each includes a first (or stationary or horizontal) portion 270 and a second (or movable or vertical) portion 272, where the second portion 272 is movable relative to (or along) the first portion 270. For example, the second portion 272 of the shelves 210 may be individually and independently movable between an extended position 220 (which is illustrated in FIGS. 3A and 3D) and an intermediate position 224 (which is illustrated in FIG. 3C) and between the intermediate position 224 and the retracted position 222 (which is illustrated in FIGS. 3B and 3E) and between the extended position 220 and the retracted position 222. In the extended position 220, a first space 252 may be defined between the second portion 272 of the shelf 210 and the support 112. In the retracted position 222, a second space 254 that may be smaller than the first space 252 may be defined between the second portion 272 of the shelf 210 and the support 112. In the intermediate position 224, a third space 256 that may be smaller than the first space 252 and larger than the second space 254 may be defined between the second portion 272 of the shelf 210 and the support. In at least one example embodiment, the second portion 272 of each shelf 210 may be slidable relative to respective first portion 270.

In at least one example embodiment, the shelves 210 may be locked in the extended position 220 during freezing of the composite (or during receipt of a frozen composite), and while in the unlocked position, the shelves 210 (and more specifically, the second portion 272 of the shelves 210) may be permitted to move in response to the thawing state of the composite. For example, although not illustrated, it should be appreciated that, in various example embodiments, the shelfing structure 200 may include one or more locking mechanisms as selected by the skilled artisan to hold the shelves 210 (and more specifically, the second portion 272 of the shelves 210) in the extended position. In at least one example embodiment, the one or more locking mechanisms may include a latching mechanism.

The shelves 210 (and more specifically, the second portion 272 of the shelves 210) of the shelfing structure 200 may be locked in the extended position 220 to receive the first bag 132 in a frozen state or to receive the first bag 132 holding a composite to be frozen. At the beginning, or during, thawing of the frozen state, the shelves 210 (and more specifically, the second portion 272 of the shelves 210) may be unlocked, manually or using automatic assembly, and allowed to move from the extended position 220 to the retreated position 222 and/or form the extended position 220 to the intermediate position 224 and/or from the intermediate position 224 to the retracted position 222. As the frozen state thaws and the shelves 210 (and more specifically, the second portion 272 of the shelves 210) may move from the extended position 220 to the retracted position 222 and/or from the extended position 220 to the intermediate position 224 and/or form the intermediate position 224 to the retracted position 222, the shelves 210 (and more specifically, the second portion 272 of the shelves 210) may apply a force or pressure to the first bag 132 that is sufficient to cause the movement of thawed liquid from the first bag 132 to the second bag 134, for example, via the tube length 138. In this manner, the relative state of the first bag 132 may control the relative position (or state) of the respective shelf 210.

FIGS. 4A-4C are illustrations of an individual shelf 210 of the shelving structure 200, where in FIG. 4A the shelf 210 (and more specifically, the second portion 272 of the shelf 210) is in the extended position 220 where the shelf 210 is associated with (e.g., supports) the first bag 132 and the first bag 132 includes a frozen composite, in FIG. 4B the shelf 210 (and more specifically, the second portion 272 of the shelf 210) is in the intermediate position 224 where the shelf 210 is associated with (e.g., supports) the first bag 132 and the first bag 132 includes a frozen composite during a thawing process, and in FIG. 4C the shelf 210 (and more specifically, the second portion 272 of the shelf 210) is in the retracted position 222 where the shelf 210 is associated with (e.g., supports) the first bag 132 and the first bag 132.

In various aspects, bag assemblies for use during the freezing and thawing of a composite including one or more components, for example, using the example shelfing structure 100 as illustrated in FIGS. 1A-1E and/or the shelfing structure 200 as illustrated in FIGS. 3A-3E, are provided.

FIG. 5A is an illustration of an example bag assembly 300 for use during the freezing and thawing of a composite including one or more components, for example, for use with the example shelfing structure 100 illustrated in FIGS. 1A-1E and/or the example shelfing structure 200 illustrated in FIGS. 3A-3E.

The bag assembly 300 may include a first bag (or container) 332 and a second bag (or container) 334. The first bag 332 may include a collection bag (or container) that is configured to receive a composite from a source (e.g., donor, patient, subject, or source). For example, in at least one example embodiment, the first bag 332 may include a plasma source bag (or container). The second bag 334 may include a freeze, thaw bag (or container) configured to receive the composite prior to freezing and component isolation. The second bag 334 may be used as a source bag (e.g., first bag 132) when used with the shelfing structure 100 as illustrated in FIGS. 1A-1E and/or the shelfing structure 200 as illustrated in FIGS. 3A-3E, and the first bag 332 may be used as the unselected component bag (e.g., second bag 134) when used with the shelfing structure 100 as illustrated in FIGS. 1A-1E and/or the shelfing structure 200 as illustrated in FIGS. 3A-3E. That is, the composite may be allowed to move from the first bag 332 and into the second bag 334 before freezing and during thawing non-precipitates may move from the second bag 334 into the first bag 332.

A tube length 338 may connect the first bag 332 and the second bag 334. The tube length 338 may have a first end and a second end, where the first end is fixedly or removably coupled to the first bag 332 and the second end is fixedly or removably coupled to the second bag 334. In at least one example embodiment, the first end of the tube length 338 may be formed integrally with the first bag 332. In at least one example embodiment, the second end of the tube length 338 may be formed integrally with the second bag 334.

The second bag 334 may include one or more angled portions 336 that are configured to aid in directing the contents held therein. For example, in at least one example embodiment, as illustrated, the second bag 334 may have a first end and an opposing second end, where the first end has a general funnel shape. The second end of the tube length 338 may be coupled to a portion of the second bag 334 away from the one or more angled portions 336.

The tube length 338 may include a line clamp 340. The line clamp 340 may be configured to move between a first (or open) position (or state) and a second (or closed) position (or state) to control movement through the tube length 338. In the first position, the line clamp 340 may apply no or minimal pressure to the tube length 338 such that materials may flow freely therethrough. In the second position, the line clamp 340 may apply a closing pressure to the tube length 338 to completely obscure fluid flow therethrough. The line clamp 340 may also be configured to move between the first position and/or the second position to an intermediate position to control flow rate through the tube length 338. In the intermediate position, the line clamp 340 may apply a partial pressure to the tube length 338 to only partially obscure fluid flow therethrough.

The line clamp 340 may be positioned at any point along the tube length 338. For example, in at least one example embodiment, the line clamp 340 may be disposed closer to the first end of the tube length 338 and nearer to the first bag 332 than the second bag 334. In other embodiments, the line clamp 340 may be disposed closer to the second end of the first tube length 338 and nearer to the second bag 334 than the first bag 332. In still other embodiments, the line clamp 340 may be disposed about halfway along the first tube length 338.

The first bag 332 and the second bag 334 may be joined together, for example, using the tube length 338 prior to or after introduction of the composite into the first bag 332. For example, in at least one example embodiment, the first bag 332 as joined to the second bag 334, for example, via the tube length 338, may be provided as part of a disposable set for use with an automated blood collection system, like the blood collection systems detailed in U.S. Pat. No. 10,585,085, titled COLLECTING COMPONENTS OF A FLUID and issued Mar. 10, 2020 and/or U.S. Pat. No. 9,758,764, titled SEPARATING COMPOSITE LIQUIDS and issued Sep. 12, 2017 and/or U.S. Pat. No. 10,618,060, titled CENTRIFUGE SAFETY MECHANISM and issued Apr. 14, 2020 and/or U.S. Pat. No. 10,166,322, titled GAIN IN SEPARATION PROCESSES WITH CONTROL LOOP and issued Jan. 1, 2019 and/or U.S. Pat. No. 9,440,011, titled HYBRID BLOOD COMPONENT STORAGE BAG AND METHOD OF MAKING SUCH BAG and issued Sep. 13, 2016 and/or U.S. Pat. No. 8,523,750, titled METHOD AND APPARATUS FOR EXTRACTING PLATELETS WITH REDUCED PLASMA CARRYOVER and issued Sep. 3, 2013 and/or U.S. Pat. No. 8,123,713, titled SYSTEM AND METHODS FOR COLLECTING PLASMA PROTEIN FRACTIONS FROM SEPARATED BLOOD COMPONENTS and issued Feb. 28, 2012 and/or U.S. Pat. No. 7,780,618, titled EXTRACORPOREAL BLOOD PROCESSING APPARATUS AND METHODS WITH PRESSURE SENSING and issued Aug. 24, 2010, the entire disclosures of which are hereby incorporated by reference. In at least one example embodiment, the first bag 332 and the second bag 334 may be provided as a single cryoprecipitate unit or set

In at least one example embodiment, the first bag 332 may be joined to the second bag 334, for example, via the tube length 338, using a tube welding mechanism after movement of the composite into the first bag 332 using, for example, an automated blood collection system. After the movement of the composite into the first bag 332, the first bag 332 and the second bag 334 may be joined using tube welding mechanisms, like the tube welding mechanisms detailed in U.S. App. 63/455,873 as filed Mar. 30, 2023, titled AUTOMATIC WELD/RESET MOTION OF CLAMPS AFTER CLAMP CLOSING/OPENING AND TUBE REMOVAL, and listing James Ladtkow as inventor and/or U.S. App. No. 63/455,840 as filed Mar. 30, 2023, titled SYSTEMS FOR AUTOMATIC OPENING OF WELDED TUBES, and listing Dennis Bebie, James Ladtkow, and Charles Hake as inventors and/or U.S. App. No. 63/459,065 as filed Apr. 13, 2023, titled CLAMP ARRANGEMENT AND PRECISION ALIGNMENT MECHANISM FOR TUBE WELDING MACHINES, and listing Filip Paeps, Bijesh Thoduvayil, Bravish Babu M, Shinu Nair, Maarten Roelandt, Lucas Vandekerckhove, and Joachim Bortels as inventors, the entire disclosures of which are hereby incorporated by reference.

FIG. 5B is an illustration of another example bag assembly 400 for use during the freezing and thawing of a composite including one or more components, for example, for use with the example shelfing structure 100 illustrated in FIGS. 1A-1E and/or the example shelfing structure 200 illustrated in FIGS. 3A-3E.

The bag assembly 400 may include two or more subassemblies, where each of the subassemblies includes a first bag (or container) 432 and a second bag (or container) 434. For example, in at least one example embodiment, as illustrated, the bag assembly 400 may include five subassemblies. Each of the individual subassemblies may be similar to the bag assembly 300 illustrated in FIG. 5A.

The first bag 432 may include a collection bag (or container) that is configured to receive a composite from a source (e.g., donor, patient, subject, or source). For example, in at least one example embodiment, the first bag 432 may include a plasma source bag (or container). The second bag 434 may include a freeze, thaw bag (or container) configured to receive the composite prior to freezing and component isolation. The second bag 434 may be used as a source bag (e.g., first bag 132) when used with the shelfing structure 100 as illustrated in FIGS. 1A-1E and/or the shelfing structure 200 as illustrated in FIGS. 3A-3E, and the first bag 432 may be used as the unselected component bag (e.g., second bag 134) when used with the shelfing structure 100 as illustrated in FIGS. 1A-1E and/or the shelfing structure 200 as illustrated in FIGS. 3A-3E. That is, the composite may be allowed to move from the first bag 432 and into the second bag 434 before freezing and during thawing non-precipitates may move from the second bag 434 into the first bag 432.

A first tube length 438 may connect the first bag 432 and the second bag 434. The first tube length 438 may have a first end and a second end, where the first end is fixedly or removably coupled to the first bag 432 and the second end is fixedly or removably coupled to the second bag 434. In at least one example embodiment, the first end of the tube length 438 may be formed integrally with the first bag 432. In at least one example embodiment, the second end of the first tube length 438 may be formed integrally with the second bag 434.

The second bag 434 may include one or more angled portions 436 that are configured to aid in directing the contents held therein. For example, in at least one example embodiment, as illustrated, the second bag 434 may have a first end and an opposing second end, where the first end has a general funnel shape. The second end of the first tube length 438 may be coupled to a portion of the second bag 434 away from the one or more angled portions 436.

The first tube length 438 may include a first line clamp 440. The first line clamp 440 may be configured to move between a first (or open) position (or state) and a second (or closed) position (or state) to control movement through the first tube length 438. In the first position, the first line clamp 440 may apply no or minimal pressure to the first tube length 438 such that materials may flow freely therethrough. In the second position, the first line clamp 440 may apply a closing pressure to the first tube length 438 to completely obscure fluid flow therethrough. The line clamp 440 may also be configured to move between the first position and/or the second position to an intermediate position to control flow rate through the tube length 438. In the intermediate position, the line clamp 440 may apply a partial pressure to the tube length 438 to only partially obscure fluid flow therethrough.

The first line clamp 440 may be positioned at any point along the tube length 438. For example, in at least one example embodiment, the first line clamp 440 may be disposed closer to the first end of the tube length 438 and nearer to the first bag 432 than the second bag 432. In other embodiments, the first line clamp 440 may be disposed closer to the second end of the first tube length 438 and nearer to the second bag 434 than the first bag 434. In still other embodiments, the first line clamp 440 may be disposed about halfway along the first tube length 438.

Although each of the individual subassemblies is illustrated as including a distinct line clamp, it should be appreciated that, in various example embodiments, a single clamp or clamping structure, for example, such as integrated in to a shelfing structure (such as the shelfing structure 100 as illustrated in FIGS. 1A-1E and/or the shelfing structure 200 as illustrated in FIGS. 3A-3E) supporting the bag assembly 400, may apply a clamping force to each of the subassemblies simultaneously.

The first bag 432 and the second bag 434 may be joined together, for example, using the first tube length 438 prior to or after introduction of the composite into the first bag 432. For example, in at least one example embodiment, the first bag 432 as joined to the second bag 434, for example, via the first tube length 438, may be provided as part of a disposable set for use with an automated blood collection system. In at least one example embodiment, the first bag 432 and the second bag 434 may be provided as a single cryoprecipitate unit or set. In at least one example embodiment, the first bag 432 may be joined to the second bag 434, for example, via the first tube length 438, using a tube welding mechanism after movement of the composite into the first bag 432 using, for example, an automated blood collection system. After the movement of the composite into the first bag 432, the first bag 432 and the second bag 434 may be joined using tube welding mechanisms.

The bag assembly 400 may also include a third bag 442 that is joined to each of the subassemblies. For example, in at least one example embodiment, as illustrated, the third bag 442 may be joined to the second bag 434 of each subassembly. More particularly, the third bag 442 may be joined to the first end of each second bag 434, where the second end includes the one or more angled portions 436. The supporting shelfing structure (such as the shelfing structure 100 as illustrated in FIGS. 1A-1E and/or the shelfing structure 200 as illustrated in FIGS. 3A-3E) may include one or more support members (e.g., hooks), disposed, for example, at an end of the individual shelves (e.g., selves 110 as illustrated in FIGS. 1A-1E and/or selves 210 as illustrated in FIGS. 3A-3E), that are configured to receive or support such third bags 442.

One or more tube lengths may fixedly or removably couple the different subassemblies and the third bag 442. For example, in at least one example embodiment, a second tube length 444 may join together each of the different subassemblies and the third bag 442. The second tube length 444 may have a first end and an opposing second end, where the first end may be fixedly or removably coupled to the second bag 434 and the second end may be fixedly or removably coupled to the third bag 442. In at least one example embodiment, the second tube length 444 may be formed integrally with the second bag 434. In at least one example embodiment, the second tube length 444 may be formed integrally with the third bag 442. Or alternatively, as illustrated, the bag assembly 400 may further include a third tube length 446 and the first end of the second tube length 444 may be fixedly or removably coupled to the second bag 434 while the second end of the second tube length 444 may be fixedly or removable coupled to a first end of the third tube length 446. The third tube length 446 may include a second end opposing the first end, where the second end is fixedly or removably coupled to the third bag 442. In at least one example embodiment, the first end of the third tube length 446 may be integrally formed with the two or more second tube lengths 444. In at least one example embodiment, the second end of the third tube length 446 may be integrally formed with the third bag 442.

The second bag 434 and the third bag 432 may be joined together, for example, using the second tube length 444 and/or the third tube length 446 prior to or after introduction of the composite into the first bag 432 and/or the second bag 434. For example, in at least one example embodiment, the second bag 434 as joined to the third bag 442, for example, via the second tube length 444 and/or the third tube length 446, may be provided as part of a disposable set for use with an automated blood collection system. In at least one example embodiment, the third bag 442 may be joined to the second bag 434, for example, via the second tube length 444 and/or the third tube length 446, using a tube welding mechanism after movement of the composite into the first bag 432 using, for example, an automated blood collection system. After the movement of the composite into the first bag 432, the first bag 432 and the second bag 434 and/or the second bag 434 and the third bag 442 may be joined using tube welding mechanisms. In at least one example embodiment, the first bag 432 and the second bag 434 may be provided as a single cryoprecipitate unit or set. In at least one example embodiment, the second bag 534 and the third bag 442 may be provided as a single cryoprecipitate unit or set. In at least one example embodiment, the first bag 432, the second bag 434, and the third bag 442 may be provided as a single cryoprecipitate unit or set.

One of the second tube length 444 and the third tube length 446 includes a second line clamp 448 that can be used to control flow in the bag assembly 400. For example, in at least one example embodiment, as illustrated, the third tube length 446 may include a second line clamp 448. The second line clamp 448 may be configured to move between a first (or open) position (or state) and a second (or closed) position (or state) to control movement through the second tube length 444. In the first position, the second line clamp 448 may apply no or minimal pressure to the third tube length 446. In the second position, the second line clamp 448 may apply a closing pressure to the first tube length 446 to completely obscure fluid flow therethrough. The second line clamp 448 may also be configured to move between the first position and/or the second position to an intermediate position to control flow rate through the tube length 446. In the intermediate position, the second line clamp 448 may apply a partial pressure to the tube length 446 to only partially obscure fluid flow therethrough.

The second line clamp 448 may be positioned at any point along the third tube length 446. For example, in at least one example embodiment, the second line clamp 448 may be disposed closer to the first end of the third tube length 446 and nearer to the second bag 434 than the third bag 422. In other embodiments, the second line clamp 448 may be disposed closer to the second end of the third tube length 446 and nearer to the third bag 442 than the second bag 434. In still other embodiments, the second line clamp 448 may be disposed about halfway along the third tube length 446.

FIG. 5C is an illustration of another example bag assembly 500 for use during the freezing and thawing of a composite including one or more components, for example, for use with the example shelfing structure 100 illustrated in FIGS. 1A-1E and/or the example shelfing structure 200 illustrated in FIGS. 3A-3E.

The bag assembly 500 may include one or more subassemblies, where each of the subassemblies includes a first bag (or container) 532 and a second bag (or container) 534. Each of the individual subassemblies may be similar to the bag assembly 300 illustrated in FIG. 5A, where the subassemblies of the bag assembly 500 further include a sedimentation bag 550.

The bag assembly 500 may include a first bag (or container) 532 and a second bag (or container) 534. The first bag 532 may include a collection bag (or container) that is configured to receive a composite from a source (e.g., donor, patient, subject). For example, in at least one example embodiment, the first bag 532 may include a plasma source bag (or container). The second bag 534 may include a freeze, thaw bag (or container) configured to receive the composite prior to freezing and component isolation. The second bag 534 may be used as a source bag (e.g., first bag 132) when used with the shelfing structure 100 as illustrated in FIGS. 1A-1E and/or the shelfing structure 200 as illustrated in FIGS. 3A-3E, and the first bag 532 may be used as the unselected component bag (e.g., second bag 134) when used with a shelfing structure 100 as illustrated in FIGS. 1A-1E and/or the shelfing structure 200 as illustrated in FIGS. 3A-3E. That is, the composite may be allowed to move from the first bag 532 and into the second bag 534 before freezing and during thawing non-precipitates may move from the second bag 534 into the first bag 532.

The bag assembly 500 may include a third bag (or container) 550 disposed between the first bag 532 and the second bag 534. The third bag 550 may be a sedimentation bag (or container). For example, the third bag 150 may be configured to capture a portion or allotment of the composite as the composite, or portions thereof, move from the first bag 532 to the second bag 534 and/or from the second bag 534 to the first bag 532. In at least one example embodiment, the third bag 150 may be selected to capture a first portion of the composite to melt, which is likely rich in protein, at the start of a thawing cycle. In at least one example embodiment, the third bag 150 may include filter (e.g., a mesh filter) that is selected to capture solutes that remain unprecipitated. In at least one example embodiment, the third bag 150 may be fabricated using a thermal isolation material.

A first tube length 538 may connect the first bag 532 and the third bag 550. The first tube length 538 may have a first end and a second end, where the first end is fixedly or removably coupled to the first bag 532 and the second end is fixedly or removably coupled to the third bag 550. In at least one example embodiment, the first end of the first tube length 538 may be formed integrally with the first bag 532. In at least one example embodiment, the second end of the first tube length 538 may be formed integrally with the third bag 550.

A second tube length 542 may connect the third bag 550 and the second bag 534. The second tube length 542 may have a first end and a second end, where the first end is fixedly or removably coupled to the third bag 550 and the second end is fixedly or removably coupled to the second bag 534. In at least one example embodiment, the first end of the second tube length 542 may be formed integrally with the third bag 550. In at least one example embodiment, the second end of the second tube length 542 may be formed integrally with the second bag 534.

The second bag 534 may include one or more angled portions 536 that are configured to aid in directing the contents held therein. For example, in at least one example embodiment, as illustrated, the second bag 534 may have a first end and an opposing second end, where the first end has a general funnel shape. The second end of the second tube length 542 may be coupled to a portion of the second bag 534 away from the one or more angled portions 536.

At least one of the first tube length 538 and the second tube length 542 may include one or more line clamps configured to move between a first (or open) position (or state) and a second (or closed) position (or state) to control movement through the respective tube length. For example, in at least one example embodiment, as illustrated the first tube length 538 may include a line clamp 540. The line clamp 540 may be configured to move between a first (or open) position (or state) and a second (or closed) position (or state) to control movement through the first tube length 538. In the first position, the line clamp 540 may apply no or minimal pressure to the first tube length 538 such that materials may flow freely therethrough. In the second position, the line clamp 540 may apply a closing pressure to the first tube length 538 to completely obscure fluid flow therethrough. The line clamp 540 may also be configured to move between the first position and/or the second position to an intermediate position to control flow rate through the first tube length 538. In the intermediate position, the line clamp 540 may apply a partial pressure to the first tube length 538 to only partially obscure fluid flow therethrough.

The line clamp 540 may be positioned at any point along the first tube length 538. For example, in at least one example embodiment, the line clamp 540 may be disposed closer to the first end of the first tube length 538 and nearer to the first bag 532 than the third bag 550. In other embodiments, the line clamp 540 may be disposed closer to the second end of the first tube length 538 and nearer to the third bag 550 than the first bag 532. In still other embodiments, the line clamp 540 may be disposed about halfway along the first tube length 538.

The first bag 532 and the third bag 550 may be joined together, for example, using the first tube length 538 prior to or after the introduction of the composite into the first bag 532. For example, in at least one example embodiment, the first bag 532 as joined to the third bag 550, for example, via the first tube length 538, may be provided as part of a disposable set for use with an automated blood collection system. In at least one example embodiment, the first bag 532 and the second bag 534 may be provided as a single cryoprecipitate unit or set

The third bag 550 and the second bag 534 may be joined together, for example, using the second tube length 542 prior to or after the introduction of the composite into the first bag 532 and/or prior to or after the joining of the first and third bags 532, 550. For example, in at least one example embodiment, the second bag 534 as joined to the third bag 550 may be provided as part of a disposable set for use with an automated blood collection system. In at least one example embodiment, the second bag 534 and the third bag 550 may be provided as a single cryoprecipitate unit or set. In at least one example embodiment, the first bag 532, the second bag 534, and the third bag 550 may be provided as a single cryoprecipitate unit or set.

In at least one example embodiment, the first bag 532 may be joined to the third bag 550, for example, via the first tube length 538, using a tube welding mechanism after movement of the composite into the first bag 532 using, for example, an automated blood collection system. After the movement of the composite into the first bag 532, the first bag 532 and the third bag 550 may be joined using tube welding mechanisms.

In at least one example embodiment, the third bag 532 may be joined to the second bag 534, for example, via the second tube length 542, using a tube welding mechanism after movement of the composite into the first bag 532 and/or the third bag 532. After the movement of the composite into the first bag 532 and/or the third bag 550, the third bag 550 and the second bag 534 may be joined using tube welding mechanisms.

In various aspects, methods for the separation of one or more components from composites are provided. The methods may include using, for example, the shelfing structure 100 as illustrated in FIGS. 1A-1E and/or one or more of the bag assemblies illustrated in 5A-5C.

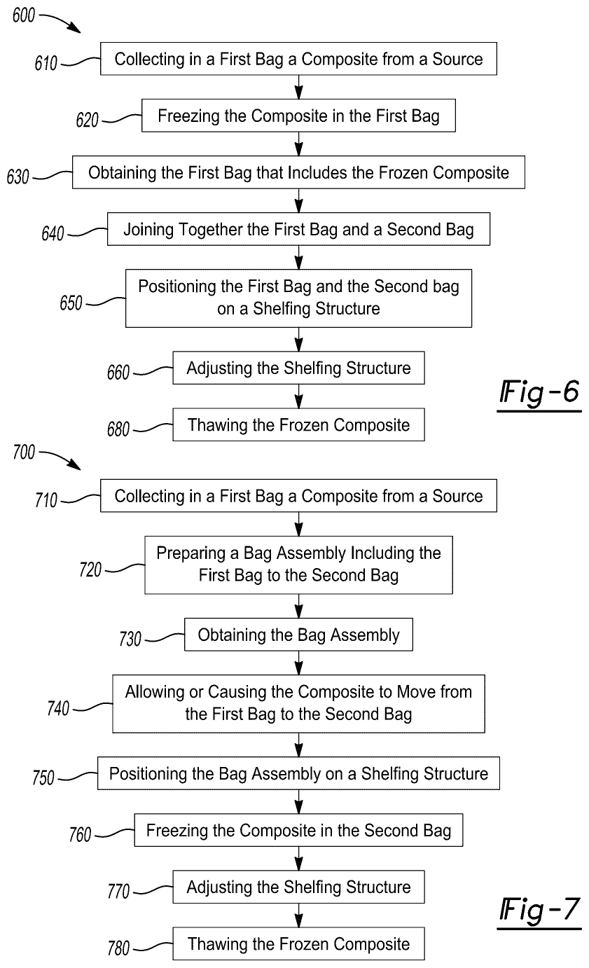

FIG. 6 is a flowchart illustrating an example method 600 for the separation of one or more components from a composite using, for example, the example shelfing structure 100 illustrated in FIGS. 1A-1E and/or the example shelfing structure 200 illustrated in FIGS. 3A-3E and/or one or more of the example bag assemblies illustrated in FIGS. 5A-5C. In at least one example embodiment, the composite may include plasma, and the method 600 may be a cyroprecipitation method.

The method 600 may include obtaining 630 a first bag (for example, like the first bag 132 as illustrated in FIGS. 1A-1E and/or as illustrated in FIGS. 3A-3E) including a frozen composite, positioning 650 the first bag and a second bag on a shelfing structure (like the shelfing structure 100 as illustrated in FIGS. 1A-1E and/or the shelfing structure 200 as illustrated in FIGS. 3A-3E), and thawing 680 the frozen composite such that precipitate-poor material (e.g., cryoprecipitate-poor plasma) is separated from a precipitate (e.g., cryoprecipitate).

The positioning 650 of the first and second bags on the shelfing structure may include disposing the second bag above the first bag. In at least one example embodiment, the positioning 650 of the first and second bags on the shelfing structure may occur while the shelves of the shelfing structure are locked in a first position. Although illustrated as a single step, it should be appreciated that, in at least one example embodiment, the first bag may be positioned independently of the second bag. That is, the first bag may be associated with the shelfing structure before the second bag or the second bag may be associated with the shelfing structure before the first bag

The thawing 680 of the frozen composite may occur in freezer or refrigerator having a consistent temperature of about 4° C. For example, although not illustrated, it should be appreciated that, in various example embodiments, the method 600 may include moving the shelfing structure supporting the first and second bags in to the freezer or refrigerator having a consistent temperature of about 4° C. In at least one example embodiment, the positioning 650 of the bags on the structure may occur in the freezer or refrigerator.

The method 600 may including adjusting 660 the shelfing structure before the thawing 680. In at least one example embodiment, the adjusting 660 may include unlocking, automatically or manually, the shelves of the shelfing structure, such that the shelves are free to move from an extended position to a retracted position, such that the respective shelves may apply a force or pressure to cause the movement of thawed liquid from the first bag to the second bag. Although the adjusting 660 is illustrated as occurring before the thawing 680, it should be appreciated that, in various example embodiments, the adjusting 660 may occur before or during the thawing 680.

In at least one example embodiment, the first bag may be part of a bag assembly that includes the second bag (for example, like the second bag 134 illustrated in FIGS. 1A-1E and/or FIGS. 3A-3E). In other example embodiments, as illustrated, the method 600 may include joining 640 together the first bag and the second bag. Although joining 640 together the first bag and the second bag is illustrated as following collecting in the first bag the composite from a source and freezing the composite in the first bag, it should be appreciated that, in various example embodiments, the first and second bags may be joined before the collection and the freezing or after the collection and before the freezing or after the freezing.

In at least one example embodiment, the first bag may be part of a bag assembly that includes the second bag and further includes a third bag (for example, like the third bag 550 illustrated FIG. 5C), where the third bag is disposed between the first bag and the second bag. In other example embodiments, the first and third bags may be provided as a bag assembly, and the method 600 may further include joining together the third bag and the second bag. It should be appreciated that, in various example embodiments, joining together the third bag and the second bag may occur before the collection and the freezing or after the collection and before the freezing or after the freezing. In still other example embodiments, the second and third bags may be provided as a bag assembly, and the method 600 may further include joining together the first bag and the third bag. It should be appreciated that, in various example embodiments, joining together the first bag and the third bag may occur before the collection and the freezing or after the collection and before the freezing or after the freezing. In still other example embodiments, the method 600 may further including joining together the first bag and the third bag and the third bag and the second bag. It should be appreciated that, in various example embodiments, joining together the first bag and the third bag may occur before the collection and the freezing or after the collection and before the freezing or after the freezing and joining together the third bag and the second bag may occur before the collection and the freezing or after the collection and before the freezing or after the freezing.

In at least one example embodiment, the method 600 may include preparing 620 the frozen composite. The frozen composite may be prepared 620 by placing the first bag including the composite in a freezer for a period of time. In at least one example embodiment, the first bag including the composite may be placed within a mold prior to freezing to control the shape of the frozen composite. In at least one example embodiment, the preparation 620 of the frozen composite may occur in a freezer or refrigerator having a consistent temperature of about 0° C. For example, although not illustrated, it should be appreciated that, in various example embodiments, the method 600 may include moving the shelfing structure supporting the first and second bags to the freezer or refrigerator having a consistent temperature of about 0° C. and/or subsequently moving the shelfing structure supporting the first and second bags to the freezer or refrigerator having a consistent temperature of about 4° C. for thawing and/or altering the temperature of a single freezer or refrigerator.

In at least one example embodiment, the method 600 may include collecting 610 the composite from a source, such as a donor, a subject, or a patient.

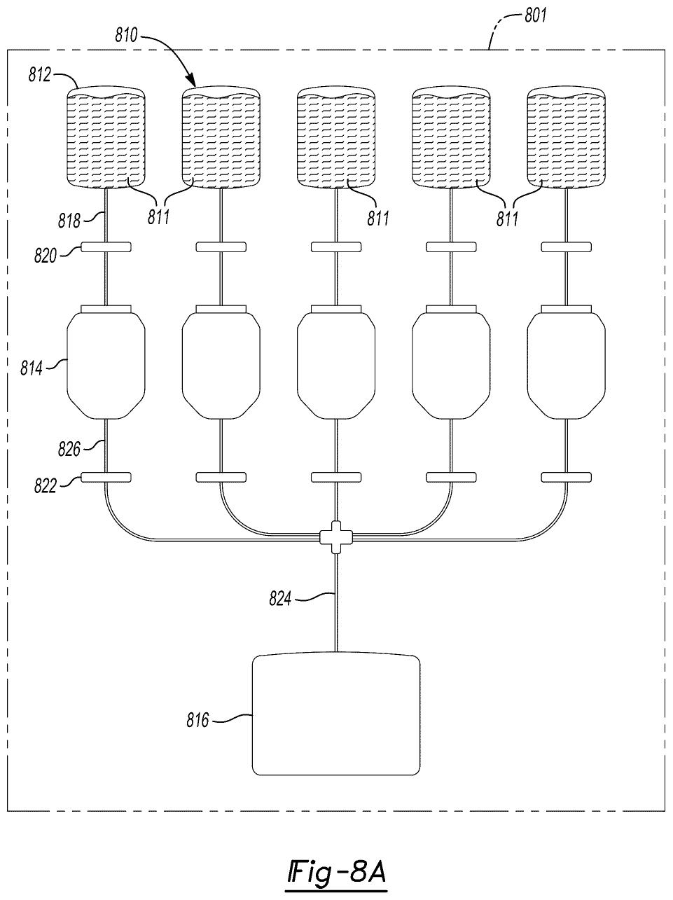

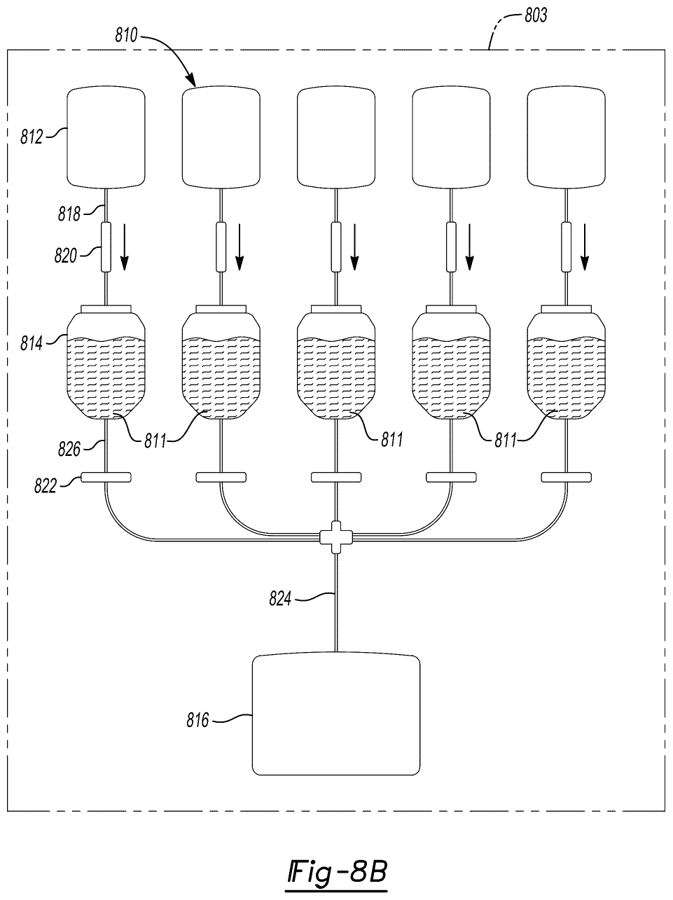

FIG. 7 is a flowchart illustrating another example method 700 for the separation of one or more components from a composite using, for example, the example shelfing structure 100 illustrated in FIGS. 1A-IE and/or the example shelfing structure 200 illustrated in FIGS. 3A-3E and/or one or more of the example bag assemblies illustrated in FIGS. 5A-5C. In at least one example embodiment, the composite may include plasma, and the method 700 may be a cyroprecipitation method.

The method 700 includes obtaining 730 a bag assembly (for example, like the bag assembly illustrated in FIGS. 5A-5C), where the bag assembly includes a first bag and a second bag, positioning 750 the bag assembly on a shelfing structure (like the shelfing structure 100 as illustrated in FIGS. 1A-1E and/or the shelfing structure 200 as illustrated in FIGS. 3A-3E), and thawing 780 a frozen composite held by the second bag such that precipitate-poor material (e.g., cryoprecipitate-poor plasma) is separated from a precipitate (e.g., cryoprecipitate).

In at least one example embodiment, the positioning 750 of the first and second bags on the shelfing structure may include disposing the first bag above the second bag. The positioning 750 of the first and second bags on the shelfing structure may occur while the shelves of the shelfing structure are locked in a first position. Although illustrated as a single step, it should be appreciated that, in at least one example embodiment, the first bag may be positioned independently of the second bag. That is, the first bag may be associated with the shelfing structure before the second bag or the second bag may be associated with the shelfing structure before the first bag.

The method 700 may include preparing 720 the bag assembly. In at least one example embodiment, the preparation 720 of the bag assembly may include joining together the first bag and a second bag using, for example, a tube length having a first end and an opposing second end, where the first end of the tube length is coupled to, or formed integrally with, the first bag and the second end of the tube length is coupled to, or formed integrally with, the second bag. In at least one example embodiment, the first bag and the second bag may be joined using a tube welding mechanism.

In at least one example embodiment, the bag assembly may include a third bag. The third bag may be a sedimentation bag disposed between the first bag and the second bag. In such instances, the preparation 720 of the bag assembly may include joining together the first bag and the third bag using, for example, a first tube length having a first end and an opposing second end, where the first end of the first tube length is coupled to, or formed integrally with, the first bag and the second end of the first tube length is coupled to, or formed integrally with, the second bag. In at least one example embodiment, the first bag and the third bag may be joined using a tube welding mechanism. The preparation 720 of the bag assembly may include joining together the third bag and the second bag using, for example, a second tube length having a first end and an opposing second end, where the first end of the second tube length is joined to, or formed integrally with, the third bag and the second end of the second tube length is joined to, or formed integrally with, the second bag. In at least one example embodiment, the third bag and the second bag may be joined using a tube welding mechanism.

In at least one example embodiment, the bag assembly may include one or more subassemblies joined to a single pooling bag, where each of the one or more subassemblies includes a first bag and a second bag. In such instances, the preparation 720 of the bag assembly may include joining together the first and second bags of the respective subassemblies. A first tube length may be used to joined together the first and second bags. The first tube length may have a first end and an opposing second end, where the first end of the first tube length is coupled to, or formed integrally with, the first bag and the second end of the second tube length is coupled to, or formed integrally with, the second bag. In at least one example embodiment, the first bag and the second bag may be joined using a tube welding mechanism. The preparation 720 of the bag assembly may include joining together the one or more subassemblies to the third bag using, for example, one or more second tube lengths and a third tube length. Each of the second tube lengths may include a first end and an opposing second end, where the first end of the respective second tube length is coupled to, or formed integrally with, one of the one or more subassemblies and the second end of the respective second tube length is coupled to, or formed integrally with, a first end of the third tube length, where a second opposing end of the third tube length is coupled to, or formed integrally with, the third bag. More particularly, the second bag may have a first side and an opposing second side, where the first side is joined to the first bag and the second side is joined to the first end of the second tube length. In at least one example embodiment, the second bag may be joined to the second tube length using a tube welding mechanism. In at least one example embodiment, the second tube length and the third tube length may be joined using a tube welding mechanism. In at least one example embodiment, the third bag and the third tube length may be joined using a tube welding mechanism.

The method 700 may include collecting 710 in a first bag a composite from a source, such as a donor, a subject, or a patient. Although the collection 710 of the composite is illustrated as preceding the preparation 720 of the bag assembly, it should be appreciated that, in various example embodiments, the collection 710 may occur after the preparation 720 of the bag assembly. In other words, the bag assembly may be prepared 720 before the collection 710 of the composite. Although the collection 710 of the composite is illustrated as preceding the obtaining 730 of the bag assembly, it should be appreciated that, in various example embodiments, the bag assembly may be obtained 730 before the collection.

The method 700 may include allowing or causing 740 the composite to move or flow from the first bag to the second bag. In at least one example embodiment, the tube length joining together the first bag and the second bag may include a line clamp, and causing 740 the composite to move from the first bag to the second bag may include changing the positioning of the line clamp to permit movement of the composite through the connecting tube length. Although causing 740 the composite to move from the first bag to the second bag is illustrated as following obtaining 730 the bag assembly, it should be appreciated that, in various example embodiments, the composite may be moved from the first bag to the second bag before the bag assembly is obtained.

The method 700 may include preparing 760 the frozen composite. In at least one example embodiment, the frozen composite may be prepared 760 by placing the second bag including the composite in a freezer for a period of time. In at least one example embodiment, the second bag including the composite may be placed within a mold prior to freezing to control the shape of the frozen composite. In at least one example embodiment, the preparation 760 of the frozen composite may occur in a freezer or refrigerator having a consistent temperature of about 0° C. For example, although not illustrated, it should be appreciated that, in various example embodiments, the method 700 may include moving the shelfing structure supporting the first and second bags to the freezer or refrigerator having a consistent temperature of about 0° C. and/or subsequently moving the shelfing structure supporting the first and second bags to the freezer or refrigerator having a consistent temperature of about 4° C. for thawing and/or altering the temperature of a single freezer or refrigerator. Although, the preparation 760 of the frozen composite is illustrated as occurring after the positioning 750 of the bag assembly on the shelfing structure, it should be appreciated that, in various example embodiments, the frozen composite may be prepared before placement of the position 750 of the bag assembly on the shelfing structure.

The method 700 may include adjusting 770 the shelfing structure before the thawing 780. In at least one example embodiment, the adjusting 770 may include unlocking, automatically or manually, the shelves of the shelfing structure, such that the shelves are free to move from an extended position to a retracted position, such that the respective shelves may apply a force or pressure to cause the movement of thawed liquid from the first bag to the second bag. Although the adjusting 770 is illustrated as occurring before the thawing 780, it should be appreciated that, in various example embodiments, the adjusting 770 may occur before or during the thawing 780.

Methods for pooling one or more components as separated from one or more composite are provided. The method may include using, for example, the shelfing structure 100 as illustrated in FIGS. 1A-1E and/or the shelfing structure 200 as illustrated in FIGS. 3A-1E and/or one or more of the bag assemblies illustrated in FIGS. 5A-5C.

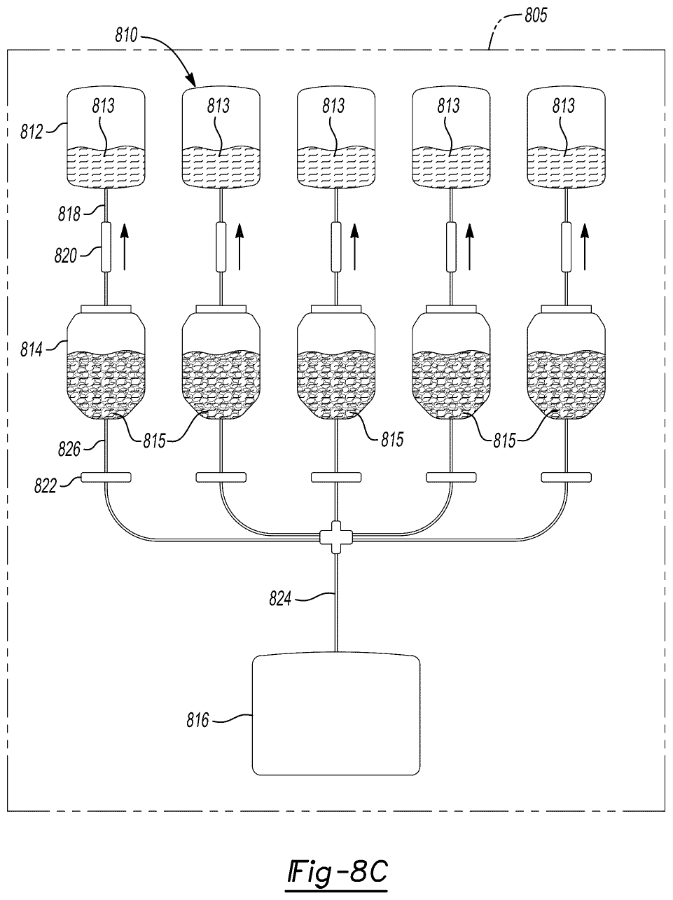

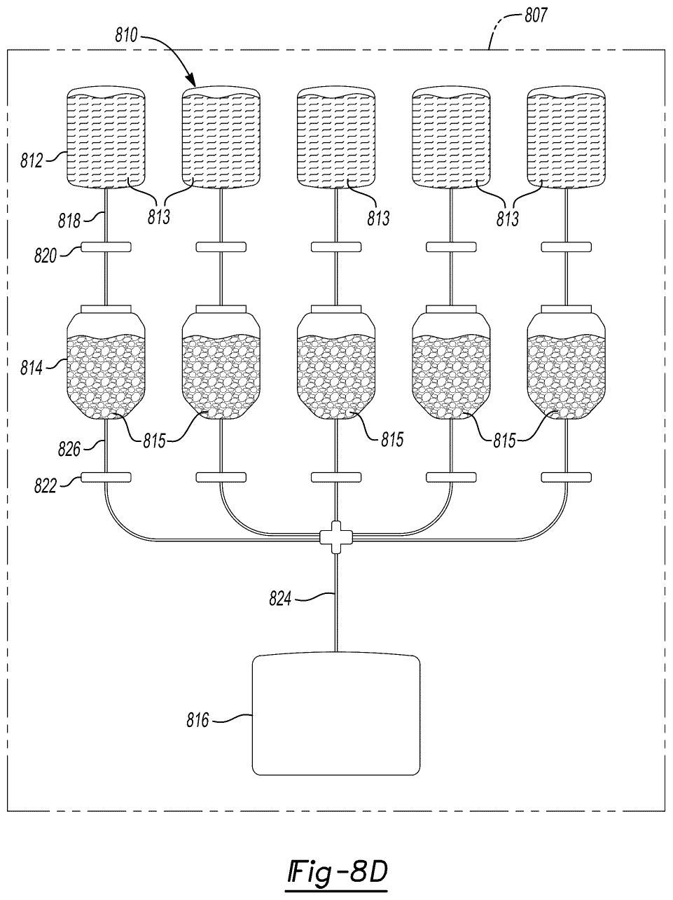

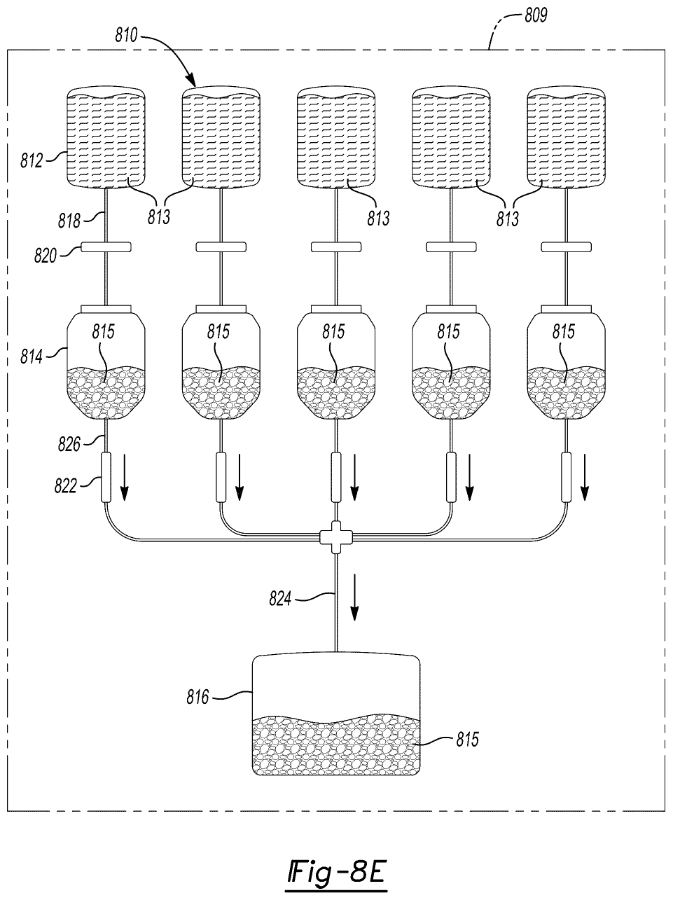

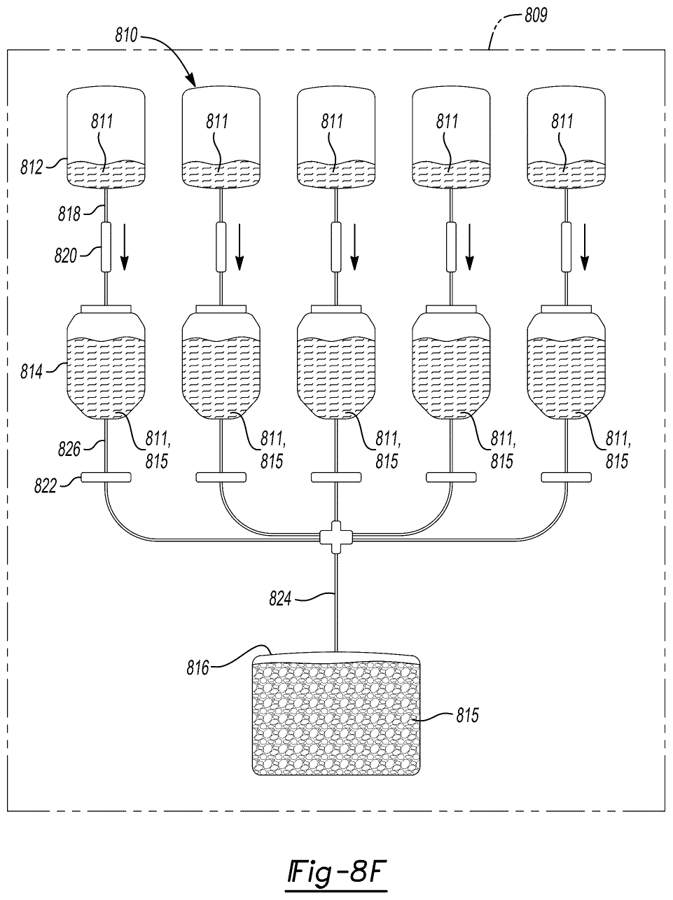

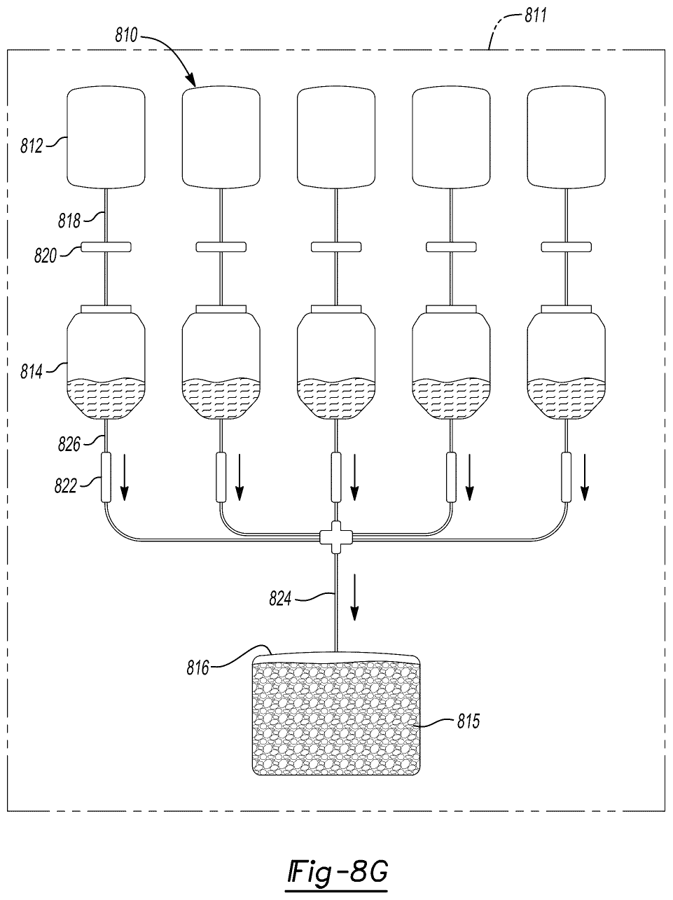

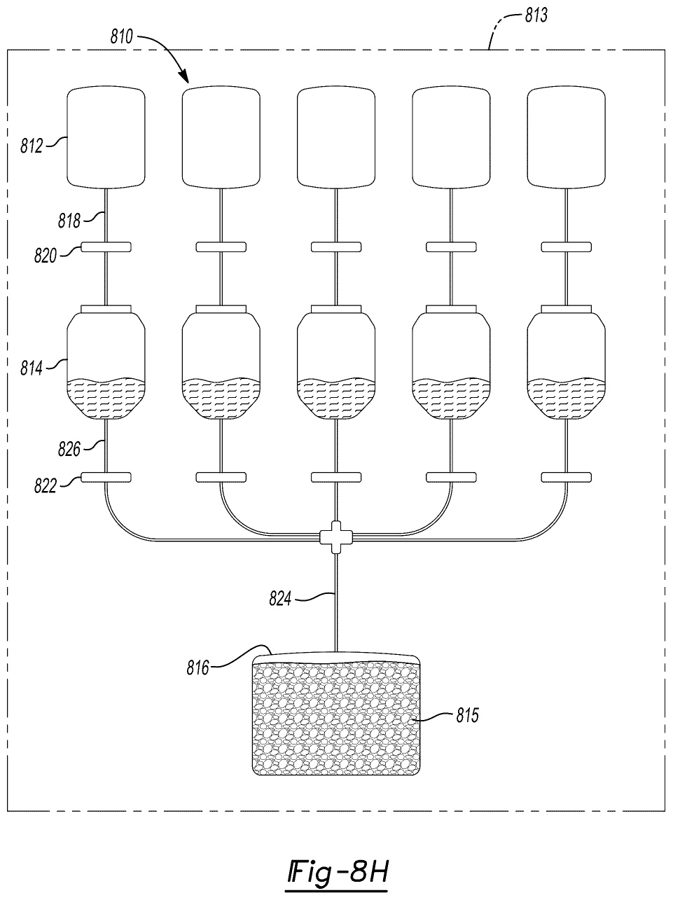

FIGS. 8A-8H are pictorials of an example method for pooling a separated precipitate using, for example, the example structure illustrated in FIGS. 1A-IE and/or the shelfing structure 200 as illustrated in FIGS. 3A-11E and/or one or more of the example bag assemblies illustrated in FIGS. 5A-5C and/or the example methods illustrated in FIGS. 6 and 7. In at least one example embodiment, the precipitate may include cryoprecipitate.

As illustrated in FIG. 8A, the method includes obtaining 801 a bag assembly 810, where the bag assembly 810 includes two or more subassemblies, each of the two or more subassemblies including a first bag (which may be like the first bag 332 illustrated in FIG. 5A and/or the first bag 432 illustrated in FIG. 5B and/or the first bag 532 illustrated in FIG. 5C) and a second bag 814 (which may be like the second bag 334 illustrated in FIG. 5A and/or the second bag 434 illustrated in FIG. 5B and/or the second bag 534 illustrated in FIG. 5C). In at least one example embodiment, as illustrated, the bag assembly 810 may include five subassemblies, where each subassembly includes a first bag 812 and a second bag 814.

Each of the subassemblies includes a first tube length 818, where the first tube length 818 has a first end and an opposing second end, the first end being coupled to, or integrally formed with, the first bag 812 and the second end being coupled to, or integrally formed with, the second bag 814. In at least one example embodiment, as illustrated, the first tube length 818, in each instance, may include a first line clamp 820. The first line clamp 820 may be configured to move between a first (or open) position (or state) and a second (or closed) position (or state) to control movement through the first tube length 820. In the first position, the first line clamp 820 may apply no or minimal pressure to the first tube length 818 such that materials may flow freely therethrough. In the second position, the first line clamp 820 may apply a closing pressure to the first tube length 818 to completely obscure fluid flow therethrough. The first line clamp 820 may also be configured to move between the first position and/or the second position to an intermediate position to control flow rate through the first tube length 818. In the intermediate position, the first line clamp 820 may apply a partial pressure to the first tube length 818 to only partially obscure fluid flow therethrough. The first line clamp 820 may be positioned at any point along the first tube length 818. For example, in at least one example embodiment, the first line clamp 820 may be disposed closer to the first end of the first tube length 818 and nearer to the first bag 812 than the second bag 814. In other embodiments, the first line clamp 820 may be disposed closer to the second end of the first tube length 820 and nearer to the second bag 814 than the first bag 812. In still other embodiments, the first line clamp 820 may be disposed about halfway along the first tube length 818. Although each of the individual subassemblies is illustrated as including a distinct line clamp 820, it should be appreciated that, in various example embodiments, a single clamp or clamping structure, for example, such as integrated in to a shelfing structure supporting the bag assembly 810, may apply a clamping force to each of the subassemblies simultaneously.

The bag assembly 810 further includes a third bag 816 joined to each of the subassemblies, and more specifically, to the second bag 814 of each subassembly. One or more tube lengths may fixedly or removably couple the different subassemblies and the third bag 816. For example, in at least one example embodiment, a second tube length 826 may join together each of the different subassemblies and the third bag 816. The second tube length 826 may have a first end and an opposing second end, where the first end may be fixedly or removably coupled to the second bag 814 and the second end may be fixedly or removably coupled to the third bag 816. In at least one example embodiment, the second tube length 826 may be formed integrally with the second bag 814. In at least one example embodiment, the second tube length 826 may be formed integrally with the third bag 816. Or alternatively, as illustrated, the bag assembly 810 may further include a third tube length 824 and the first end of the second tube length 826 may be fixedly or removably coupled to the second bag 814 while the second end of the second tube length 826 may be fixedly or removable coupled to a first end of the third tube length 824. The third tube length 824 may include a second end opposing the first end, where the second end is fixedly or removably coupled to the third bag 816. In at least one example embodiment, the first end of the third tube length 824 may be integrally formed with the two or more second tube lengths. In at least one example embodiment, the second end of the third tube length 824 may be integrally formed with the third bag 816.

One of the second tube length 826 and the third tube length 824 includes a second line clamp 822 that can be used to control flow in the bag assembly 810. For example, in at least one example embodiment, as illustrated, each of the second tube length 826 as joined to the different subassemblies includes a second line clamp 822. The second line clamp 822 may be configured to move between a first (or open) position (or state) and a second (or closed) position (or state) to control movement through the second tube length 826. In the first position, the second line clamp 822 may apply no or minimal pressure to the second tube length 826. In the second position, the second line clamp 822 may apply a closing pressure to the second tube length 826 to completely obscure fluid flow therethrough. The second line clamp 822 may also be configured to move between the first position and/or the second position to an intermediate position to control flow rate through the second tube length 826. In the intermediate position, the second line clamp 822 may apply a partial pressure to the second tube length 826 to only partially obscure fluid flow therethrough. The second line clamp 822 may be positioned at any point along the second tube length 826. For example, in at least one example embodiment, the second line clamp 822 may be disposed closer to the first end of the third tube length 826 and nearer to the second bag 814 than the third tube length 824. In other embodiments, the second line clamp 822 may be disposed closer to the second end of the second tube length 826 and nearer to the third tube length 824 than the second bag 814. In still other embodiments, the second line clamp 822 may be disposed about halfway along the second tube length 826. Although each of the second tube lengths 826 is illustrated as including a distinct line clamp 822, it should be appreciated that, in various example embodiments, a single clamp or clamping structure, for example, such as integrated in to a shelfing structure supporting the bag assembly 810, may apply a clamping force to each of the subassemblies simultaneously. Although the bag assembly 810 is illustrated as including a plurality of second line clamps 822, it should be appreciated, that in at least one example embodiment, the bag assembly 810 may include a single second line clamp that is positioned, along the third tube length 824.

In at least one example embodiment, the obtaining 801 of the bag assembly 810 may include joining together the first and second bags 812, 814 of the different subassemblies, using, for example, the first tube lengths 818. In at least one example embodiment, obtaining 801 the bag assembly 810 may include joining together the different subassemblies and the third bag 816, using, for example, the second and third tube lengths 826, 824.

In at least one example embodiment, when the bag assembly 810 is obtained, the first bags 812 may include a composite 811 and the first line clamps 820 may be in a closed position. The second line clamps 822 may also be in a closed position when the bag assembly 810 is obtained. Although not illustrated, it should be appreciated that, in various example embodiments, the method may include collecting the composite 811 from a source, such as a donor, a subject, or a patient.

In at least one example embodiment, the obtaining 801 of the bag assembly 810 may include associating the bag assembly 810 with a shelfing structure (such as the shelfing structure 100 illustrated in FIGS. 1A-1E and/or the shelfing structure 200 illustrated in FIGS. 3A-3E). Associating the bag assembly 819 with the shelfing structure may include placing the first bags 812 above the second bags 814. The bag assembly 810 may be associated with the shelfing structure while the shelves of the shelfing structure are locked in a first (or extended) position.

As illustrated in FIG. 8B, the method may include allowing or causing 803 the composite 811 to move or flow from the first bags 812 to the second bags 814 of the different subassemblies. The causing 802 of the composite 811 to move from the first bags 812 to the second bags 814 may include changing the positioning of the first line clamps 820 to permit movement of the composite 811 through the connecting first tube lengths 818. In at least one example embodiment, the causing 802 of the composite 811 to move from the first bags 812 to the second bags 814 may include removing the first line clamps 820 from the bag assembly 810. Although the causing 803 of the composite 811 to move from the first bags 812 to the second bags 814 is discussed as following the association of the bag assembly 810 with the shelfing structure, it should be appreciated that, in various example embodiments, the causing 803 of the composite 811 to move from the first bags 812 to the second bags 814 may occur before or during the association of the bag assembly 810 with the shelfing structure.

Once the composite 811 is held by the second bags 814 and the bag assembly 810 is associated with the shelfing structure (such as the shelfing structure 100 illustrated in FIGS. 1A-1E and/or the shelfing structure 200 illustrated in FIGS. 3A-3E), the method may include preparing a frozen composite. The frozen composite may be prepared by placing the second bag including the composite in a freezer for a period of time. For example, in at least one example embodiment, the shelfing structure supporting the bag assembly 810 may be placed in a freezer or refrigerator having a consistent temperature of about 0° C.

As illustrated in FIG. 8C, the method may include precipitating 805 one or more components from the composite as the frozen composite is thawed. At the beginning, or during, precipitation 805, the shelves of the shelfing structure may be unlocked, manually or using automatic assembly, and allowed to move from the extended position to a retreated position and/or form the extended position to an intermediate position and/or from the intermediate position to the retracted position. The precipitation 805 includes allowing the frozen composite to melt. For example, in at least one example embodiment, the precipitation 805 may include moving the shelfing structure supporting the first and second bags to the freezer or refrigerator having a consistent temperature of about 0° C. and/or subsequently moving the shelfing structure supporting the first and second bags to the freezer or refrigerator having a consistent temperature of about 4° C. for thawing and/or altering the temperature of a single freezer or refrigerator. As the frozen state thaws and the shelves move from the extended position to the retracted position and/or from the extended position to the intermediate position and/or form the intermediate position to the retracted position, the shelves may apply a force or pressure to the second bags 814 that is sufficient to cause the movement of thawed material 813 from the second bags 814 to the respective first bag 812, for example, via the first tube lengths 818, while retaining a precipitate 815 in the second bags 814.

After the thawing (for example, after a predetermined time period and/or after a predetermined volume collection and/or position of the respective shelves of the shelfing structure), the method may include, as illustrated in FIG. 8D, adjusting 807 the first line clamps 820 to stop movement between the first bags 812 and the respective second bags 814. For example, the first line clamps 820 may be moved from the open position to the closed position. In at least example embodiment, adjusting the first line clamps 820 may include re-applying or re-introducing the first line clamps 820 to the bag assembly.

After flow is stopped between the first bags 812 and the second bags 814, the method may include, as illustrated in FIG. 8E, adjusting the second line clamps 822 may include allowing or causing 809 the precipitate 815 from each of the different second bags 814 to move or flow from the second bags 814 to the third bag 816. The causing 809 of the precipitate 815 to move from the second bags 814 to the third bags 816 may include changing the positioning of the second line clamps 822 to permit movement of the precipitate 815 through the connecting second and third tube lengths 826, 824. In at least one example embodiment, the causing 809 of the precipitate 815 to move from the second bags 814 to the third bag 816 may include removing the second line clamps 822 from the bag assembly 810. In various aspects, pooling the contents of the two or more subassemblies allows a sufficient amount of the precipitate to be collected in a single bag (i.e., the third bag 816) as may be required for adult dosing of a subject or patient for different therapies and treatments.

In at least one example embodiment, after the precipitate 815 is pooled, the method may further include, as illustrated in FIG. 8F, adjusting 810 the first and second line clamps. For example, the second line clamps 822 may be adjusted to prevent movement through from the second bags 814 of the different subassemblies to the third bag 816, and the first line clamps 820 may be adjusted to allow movement of additional material 811 from the first bags 812 to the respective secondbags 814 of the subassemblies.

After a predetermined time period and/or after a predetermined volume collection and/or position of the respective shelves of the shelfing structure following the adjusting of the first line clamps 820 to allow movement of additional material 811 from the first bags 812 to the respective second bags 814 of the subassemblies, the method may include, as illustrated in FIG. 8G, adjusting the second line clamps 822 to allow movement from the second bags of the different subassemblies to the third bag 816. This subsequent movement from the first bags 812 to the respective second bags 814 of the subassemblies and then from the second bags 814 to the third bag 816 may allow any residual amounts of the one or more components or precipitate 815 to be captured.

After a predetermined time period and/or after a predetermined volume collection and/or position of the respective shelves of the shelfing structure, the method includes, as illustrated in FIG. 8H, adjusting the second line clamps 822 to prevent movement through from the second bags 814 of the different subassemblies to the third bag 816. Although not illustrated, it should be appreciated that, in various example embodiments, the method may include removing the bag assembly 810 form the shelfing structure and/or separating the third bag 816 from the remaining of the bag assembly 810 for individual storage and/or transport.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A shelfing structure for use during precipitation of one or more components from a composite, the shelfing structure comprising:
a support; and
one or more expression plates movably coupled to the support, each of the one or more expression plates movable between a first position and a second position, and each of the one or more expression plates configured to receive one or more bags of a bag assembly.

2. The shelfing structure of claim 1, wherein the shelfing structure further includes:
one or more spring loaded hinges movably coupling each of the one or more expression plates and the support.

3. The shelfing structure of claim 1, wherein each of the one or more expression plates includes a first portion and a second portion, the second portion being movably coupled to the first portion to define the first position and the second position.

4. The shelfing structure of claim 1, wherein the shelfing structure further includes:
one or more locking mechanisms configured to maintain the one or more expression plates in the first position.

5. The shelfing structure of claim 1, wherein the support further includes a plurality of castors.

6. The shelfing structure of claim 1, wherein
each of the one or more expression plates has a first surface opposing a second surface,
a first distance between the first surface and the support defining the first position, and
a second distance between the first surface and the support that is less than the first distance defining the second position.

7. The shelfing structure of claim 6, wherein
the bag assembly includes a first bag and a second bag, and
the first surface is configured in each instance to support the first bag.

8. The shelfing structure of claim 7, wherein the first surface includes one or more guiders for positioning the first bag.

9. The shelfing structure of claim 7, wherein the second surface is configured in each instances to support the second bag.

10. The shelfing structure of claim 9, wherein the second surface includes one or more guiders for positioning the second bag.

11. The shelfing structure of claim 8, wherein the second surface is configured to support the second bag above the first bag.

12. The shelfing structure of claim 10, wherein the bag assembly further includes:
a tube length connecting the first bag and the second bag.

13. The shelfing structure of claim 12, wherein the tube length includes a line clamping member.

14. The shelfing structure of claim 12, wherein the shelfing structure further includes:
a clamping structure configured to clamp the tube length.

15. The shelfing structure of claim 6, wherein the first surface includes one or more stoppers configured to engage with the support to define the second position.

16. A bag assembly for pooling one or more components as separated from two or more composites, the bag assembly comprising:
a first bag subassembly including a first bag, a second bag, and a first tube length connecting the first bag and the second bag;
a second bag subassembly including a third bag, a fourth bag, and a second tube length connecting the third bag and the fourth bag; and
a single bag in fluid communication with the second bag of the first bag subassembly and the fourth bag of the second bag subassembly.

17. The bag assembly of claim 16, wherein
the first tube length includes a first line clamp configured to move between a first position and a second position,
in the first position the first line clamp occludes the first tube length and prevents material movement between the first bag and the second bag, and
in the second position the first line clamp allows material movement between the first bag and the second bag.

18. The bag assembly of claim 17, wherein the first bag, the first tube length, and the second bag are integrally formed.

19. The bag assembly of claim 17, wherein
the second tube length includes a second line clamp configured to move between a first position and a second position,
in the first position the second line clamp occludes the second tube length and prevents fluidic movement between the third bag and the fourth bag, and
in the second position the second line clamp allows fluidic movement between the third bag and the fourth bag.

20. The bag assembly of claim 19, wherein the third bag, the second tube length, and the fourth bag are integrally formed.

21. The bag assembly of claim 16, wherein the bag assembly further includes:
a third tube length connecting the single bag and the second bag of the first subassembly and also the single bag and the fourth bag of the second bag subassembly.

22. The bag assembly of claim 21, wherein the second bag of the first subassembly, the fourth bag of the second bag subassembly, the third tube length, and the single bag are integrally formed.

23. The bag assembly of claim 21, wherein the third tube length includes:
a third clamp configured to move between a first position and a second position,
in the first position the third clamp occludes the third tube length and prevents material movement between the single bag and the second bag of the first subassembly and the fourth bag of the second bag subassembly, and
in the second position the third clamp allows material movement between the single bag and the second bag of the first subassembly and the fourth bag of the second bag subassembly.

24. The bag assembly of claim 21, wherein the bag assembly further includes:
a fourth tube length fluidically connecting the second bag of the first subassembly and the third tube length.

25. The bag assembly of claim 24, wherein the fourth tube length includes:
a third clamp configured to move between a first position and a second position,
in the first position the third clamp occludes the fourth tube length and prevents material movement between the second bag of the first subassembly and the third tube length, and
in the second position the third clamp allows material movement between the second bag of the first subassembly and the third tube length.

26. The bag assembly of claim 24, wherein the bag assembly further includes:
a fifth tube length fluidically connecting the fourth bag of the second subassembly and the third tube length.

27. The bag assembly of claim 26, wherein the fifth tube length includes:
a fourth clamp configured to move between a first position and a second position,
in the first position the fourth clamp occludes the fifth tube length and prevents material movement between the fourth bag of the second subassembly and the third tube length, and
in the second position the fourth clamp allows material movement between the fourth bag of the second subassembly and the third tube length.

28. The bag assembly of claim 16, wherein at least one of the second bag of the first subassembly and the fourth bag of the second subassembly has as funnel shaped portion disposed towards the single bag.

29. The bag assembly of claim 16, wherein the first tube length includes a sedimentation filter.

30. A method for separating one or more components from a composite, the method comprising:
positioning a shelfing structure holding one or more bag assemblies in an environment having a predetermined or selected thawing temperature, each of the one or more bag assemblies including a first bag including the composite in a frozen state and a second bag configured to receive the one or more components, the shelfing structure configured to hold the second bag above the first bag in each instance.

31. The method of claim 30, wherein the shelfing structure includes
a support; and
one or more expression plates movably coupled to the support, each of the one of the one or more expression plates having a first surface opposing a second surface, the first surface configured to support the first bag of a first bag assembly of the one or more bag assemblies, and the second surface configured to support the second bag of a second bag assembly of the one or more bag assemblies.

32. The method of claim 31, wherein the shelfing structure further includes:
one or more spring loaded hinges that movably couple each of the one or more expression plates and the support.

33. The method of claim 31, wherein each of the one or more expression plates is movable between a first position and a second position in response to the thawing of the frozen state of the composite.

34. The method of claim 33, wherein the shelfing structure further includes one or more locking mechanisms configured to maintain the one or more expression plates in the first position, and the method further includes:
adjusting the one or more locking mechanism prior to, concurrently with, or subsequent to the positioning of the shelfing structure holding the one or more bag assemblies into the environment having the predetermined or selected thawing temperature.

35. The method of claim 30, wherein
each of the one or more bag assemblies includes a first tube length connecting the first bag and the second bag,
the first tube length including a line clamp configured to move between a first position and a second position,
in the first position the line clamp occludes the first tube length and prevents fluidic movement between the first bag and the second bag, and
the method further includes adjusting the line clamp prior to, concurrently with, or subsequent to the positioning of the structure holding the one or more bag assemblies into the environment having the predetermined or selected thawing temperature.

36. The method of claim 35, wherein the method further includes:
joining together the first bag and the second bag via the first tube length.

37. The method of claim 35, wherein
each of the one or more bag assemblies is connected to a third bag.
